(19) United States
(12) Patent Application Publication
(10) Pub. No.: US 2011/0287026 A1
(43) Pub. Date: Nov. 24, 2011

(54) SIRT4 AND USES THEREOF

(75) Inventors: Marcia Haigis, Winchester, MA (US); Vincent De Boer, Utrecht (NL)

(73) Assignee: President and Fellows of Harvard College, Cambridge, MA (US)

(21) Appl. No.: 13/120,463

(22) PCT Filed: Sep. 23, 2009
(86) PCT No.: PCT/US09/58041

§ 371 Date: Aug. 10, 2011

Related U.S. Application Data
(60) Provisional application No. 61/192,892, filed on Sep. 23, 2008.

(51) Int. Cl. A61K 39/395; C12Q 1/48; A61K 31/455; A61K 31/166; A61P 1/00; A61P 35/00; A61P 3/10; A61P 21/00; A61P 27/02; A61K 31/7088; A61K 31/4709

(52) U.S. Cl. 424/158.1; 514/44 A; 435/15; 514/355; 514/619; 514/314

(57) ABSTRACT

Provided herein are SIRT4 compositions and methods of use thereof. The invention provides functional information for use in the identification and design of compounds that modulate SIRT4 enzyme activity (e.g., inhibition of fatty acid oxidation, ADP ribosylation, and/or downregulation of glutamate dehydrogenase), and to the compounds identified by such methods and the research, diagnostic and therapeutic uses of such compounds.

### RELATED APPLICATIONS

This application claims the benefit of priority to U.S. Provisional Patent Application Ser. No. 61/192,892, filed Sep. 23, 2008, the contents of which is hereby incorporated by reference.

### BACKGROUND OF THE INVENTION

Sir2 (Silent information regulator 2) and its homologs, extend lifespan in yeast, worms and flies. Mammals contain seven homologs of sir2 (sirtuins, SIRT1-7) that possess NAD⁺-dependent deacetylase and/or ADP-ribosylation activity. SIRT1, the closest mammalian sir2 ortholog, is the most studied sirtuin and has been shown to deacetylate more than a dozen substrates to promote metabolic adaptation and cell survival. For example, in pancreatic beta cells, SIRT1 represses the expression of mitochondrial uncoupling protein and increases insulin secretion. In the liver, SIRT1 activity is up-regulated during fasting, leading to modulation of gluconeogenesis through deacetylation of FOXO1, CRTC2 and PGC-1α.

Three of the mammalian sirtuins (SIRT3, SIRT4 and SIRT5) are endogenously located in the mitochondria and may play roles as sensors of energy status in this organelle. SIRT3 deacetylates acetyl-CoA synthetase 2 (AceCS2), glutamate dehydrogenase (GDH) and complex I of the electron transport chain in vitro, but SIRT3 knockout mice do not have an obvious phenotype under basal conditions. SIRT5 possesses weak deacetylase activity, and its in vivo targets remain unidentified. In pancreatic beta cells, SIRT4 regulates the conversion of glutamate and glutamine to α-ketoglutarate by ADP-ribosylating and inhibiting GDH, thereby repressing insulin secretion from pancreatic beta cells. Nevertheless, SIRT4 is broadly expressed, and its roles in tissues other than the pancreas have not been described.

### SUMMARY OF THE INVENTION

Mitochondrial function is implicated in a wide variety of disorders, including, for example, physiological and pathophysiological stress, obesity, cardiovascular disease, aging and age-related disease. It has now been discovered that the mitochondrial protein SIRT4 is a key regulator of fatty acid oxidation and plays an important role in the context of disease, aging and associated pathologies. Suppression of SIRT4 activity prevents diet-induced weight gain by reducing adipose tissue and allows for the maintenance of a lean phenotype even under conditions of a high fat diet. Described herein are methods and compositions for the regulation of lipid metabolism, including fatty acid oxidation, the control of weight gain and the treatment of metabolic syndromes.

In a one aspect, the invention provides a method of evaluating SIRT4 fatty acid oxidation repression activity, the method comprising providing a cell-free composition comprising a SIRT4 protein, an enzyme that catalyzes fatty acid oxidation, and a substrate, and evaluating fatty acid oxidation activity in the composition. In some embodiments the substrate comprises a fatty acid. Optionally, the method also provides the step of adding a test compound to the cell-free composition. In some embodiments, the test compound is a small molecule, an antibody, or a nucleic acid.

In another aspect, the invention provides a method for measuring an inhibitory property of a test compound towards a SIRT4 protein, comprising contacting the SIRT4 protein with the test compound in the presence of an enzyme that catalyzes fatty acid oxidation, and a substrate, measuring the test rate of fatty acid oxidation in the presence of the test compound, and comparing the test rate of fatty acid oxidation with a control rate of fatty acid oxidation obtained in the absence of the test compound, where an increase in the test rate relative to the control rate is indicative of an inhibitory property of the test compound. In some embodiments, the test compound is a small molecule, an antibody, or a nucleic acid.

In a further aspect, the invention provides a method for measuring a stimulatory property of a test compound towards a SIRT4 protein, including the steps of contacting the SIRT4 protein with the test compound in the presence of an enzyme that catalyzes fatty acid oxidation, and a substrate, measuring the test rate of fatty acid oxidation in the presence of the test compound, and comparing the test rate of fatty acid oxidation with a control rate of fatty acid oxidation obtained in the absence of the test compound, where a decrease in the test rate relative to the control rate is indicative of a stimulatory property of the test compound. In some embodiments, the test compound is a small molecule, an antibody, or a nucleic acid.

In yet a further aspect, the invention provides a method of treating or preventing a fatty acid oxidation disorder (FOD) in a mammalian subject, comprising administering to the subject an effective amount of an agent that reduces SIRT4 protein activity. Exemplary FODs include obesity, Medium Chain Acyl-CoA Dehydrogenase (MCAD) Deficiency, Short Chain Acyl-CoA Dehydrogenase (SCAD) Deficiency, long-chain Acyl-CoA dehydrogenase (LCAD) deficiency, Carnitine Palmityltransferase Translocase I & II Deficiency, Carnitine acylcarnitine translocase deficiency, Very Long Chain Acyl-CoA Dehydrogenase (VLCAD) Deficiency, Glutaricaciduria II, EFT Deficiency HMG Carnitine Transport Defect (Primary Carnitine Deficiency), Long Chain 3-Hydroxyacyl-CoA Dehydrogenase (LCHAD) Deficiency, Trifunctional Protein (TFP) Deficiency, 2,4 Dienoyl-CoA Reductase Deficiency, 3-Hydroxy Acyl CoA Dehydrogenase Deficiency (HADH), Electron Transfer Flavoprotein (ETF) Dehydrogenase Deficiency, and 3-Hydroxy-3 Methylglutaryl-CoA (HMG) Lyase Deficiency. In certain embodiments, the levels of SIRT4 are modulated in a hepatocyte. In some embodiments, the agent is an antagonistic nucleic acid that reduces SIRT4 expression. In other embodiments, the agent comprises a nucleic acid that targets SIRT4 mRNA or an antibody that targets SIRT4 protein.

In another aspect, the invention provides a method of evaluating the effect of a test compound on SIRT4, the method comprising providing a reaction mixture comprising SIRT4 and a test compound, and evaluating a fatty acid oxidation activity of SIRT4. In some embodiments the test compound is a small molecule. In other embodiments, the method is repeated for each of a plurality of test compounds from a chemical library. In further embodiments, the reaction mixture is provided in a eukaryotic cell, such as a hepatocyte. In still further embodiments, the reaction mixture is provided in a mammalian subject.

In a further aspect, the invention provides a method of inducing weight gain or fatty acid deposition in a mammalian subject, comprising administering to the subject an effective amount of an agent that increases SIRT4 protein activity. For example, the subject is malnourished.

In yet a further aspect, the invention provides a method of increasing an activity of a peroxisome proliferator-activated receptor-alpha (PPAR-a) in a mammalian cell, comprising contacting the mammalian cell with a compound that reduces SIRT4 activity.

In another aspect, the invention provides a method of increasing a mammalian subject's energy consumption, comprising administering to the subject a SIRT4 inhibitor. For example, the subject is overweight, is suffering from or at risk of developing a mitochondrial-related disease, or has a metabolic disorder resulting in reduced fatty acid oxidation and/or increased fatty acid deposition in the subject's tissue. Mitochondrial-related diseases include aging, MELAS syndrome, muscular dystrophy, diabetes, Leber's hereditary optic neuropathy, Leigh syndrome, NARP syndrome, and Myoneurogenic gastrointestinal encephalopathy. In some embodiments, the SIRT4 inhibitor is provided in an effective dose such that fat storage in a tissue of the subject is reduced. In other embodiments, the SIRT4 inhibitor is administered to a liver tissue, a brown adipose tissue, a skeletal muscle tissue, or a combination thereof.

In a further aspect, the invention provides a method of reducing a cholesterol level in a mammalian subject, comprising administering to the subject a SIRT4 inhibitor in an effective amount such that a cholesterol level is reduced. For example serum cholesterol level may be reduced. In some embodiments, the method also includes administering to the subject an effective amount of a peroxisome proliferator-activated receptor-alpha agonist, such as ciprofibrate, clofibrate, fenofibrate, bezafibrate, WY14,643, or a combination thereof.

In another aspect, the invention provides a method of reducing a reactive oxygen species (ROS) in a tissue, comprising contacting the tissue with a SIRT4 activator. The ROS is, for example, an oxygen ion, a free radical, or a peroxide-containing compound. In some aspects, the tissue comprises a hepatocyte.

In a further aspect, the invention provides a method of increasing SIRT1 activity in a cell comprising contacting said cell with a SIRT4 inhibitor. In some embodiments, said SIRT4 inhibitor is selected from a group consisting of a small molecule, an antibody and an antagonistic nucleic acid.

In yet a further aspect, the invention provides a composition comprising a SIRT4 inhibitor and a peroxisome proliferator-activated receptor-alpha agonist. In some embodiments, the peroxisome proliferator-activated receptor-alpha agonist is ciprofibrate, clofibrate, fenofibrate, bezafibrate, WY14,643, or a combination thereof.

### BRIEF DESCRIPTION OF THE DRAWINGS

FIG. 1 shows the results of quantitative RT-PCR assays depicting the expression of SIRT4 (FIG. 1A), SIRT3 (FIG. 1B), SIRT5 (FIG. 1C), Gk (FIG. 1D), Cpt1a (FIG. 1E) and Acot3 (FIG. 1F) in hepatocytes taken from WT mice that had been fasted for the indicated period of time.

FIG. 2 shows the results of microarray analysis of gene expression in whole liver of SIRT4 KO mice compared to SIRT4 WT mice. FIG. 2A lists the gene ontology terms over-represented in the gene expression profile of SIRT4 KO mouse livers. FIG. 2B depicts the classification of pathways and metabolic processes of all annotated, differentially expressed genes with a p-value of <0.01. FIG. 2C depicts the relative expression of genes with a p-value of <0.1 associated with lipid metabolic processes.

FIG. 3 shows the primers used in quantitative RT-PCR assays to detect expression of Acot3, Asns, Egfr, Lipg, B2m and Rsp16.

FIG. 4 shows the results of quantitative RT-PCR assays to detect the expression of cpt1a, lipg, acot3, asns, egfr, SIRT4 and esr in whole liver taken from fed or fasted SIRT4 WT or SIRT4 KO mice.

FIG. 5 shows the similarity between the SIRT4 KO liver transcriptome and published liver transcriptomes from Gene Expression Omnibus (GEO) and ArrayExpress. WY PPARα WT: WT mice treated for 5 days with WY14643 (GSE8295, (Rakhshandehroo et al., (2007) PPAR Research 2007, 26839)), WY PPARα KO: PPARα KO mice treated for 5 days with WY14643 (GSE8295 (Rakhshandehroo et al., (2007) PPAR Research 2007, 26839)), PPARα KO: WT vs. PPARα KO mice not treated with WY14643 (GSE8295, (Rakhshandehroo et al., (2007) PPAR Research 2007, 26839)), CR: Long term caloric restriction mice vs control diet (GSE2431, (Dhahbi et al., (2005) Physiol Genomics 23, 343-350)), PGC-1β mut: PGC-1β mutant mice vs. WT mice (GSE6210, (Vianna et al., (2006) Cell Metab 4, 453-464)), aging1: 22 mo vs. 4 mo WT Snell dwarf mice (GSE3129, (Boylston et al., (2004) Aging Cell 3, 283-296)), aging2: 22 mo vs. 4 mo WT Ames dwarf mice (GSE3150, (Boylston et al., (2006) AGE 28, 125-144)), aging3: 130 wks vs 13 wks WT mice (E-MEXP-1504, (Schumacher et al., (2008) PLoS Genet. 4, e1000161)). Significance was calculated using permutation. * p<0.0001.

FIG. 6 shows the results of quantitative RT-PCR assays that detect expression of PPARα and PPARα target genes in SIRT4 KO and SIRT4 WT livers.

FIG. 7A shows immunoblots depicting SIRT4 expression in primary mouse embryonic fibroblasts (MEFs) from SIRT4 KO and SIRT4 WT mice infected with control (−) or SIRT4 expression virus (+). FIG. 7B shows the expression of pdk4 in either SIRT4 KO or SIRT4 WT MEFs infected with control (−) or SIRT4 expression virus (+) and either treated or untreated with 50 μM WY14643. FIG. 7C shows the expression of pdk4 in either SIRT4 KO (−/−) or SIRT4 WT (+/+) MEFs and either treated or untreated with 50 μM WY14643.

FIG. 8A shows immunoblots depicting expression of SIRT4-Flag (T4), H161A-SIRT4-Flag (Mut), HA-PPARα and actin in transiently transfected human embryonic kidney 293T (HEK293T) cells co-transfected with a luciferase reporter driven by three tandem repeats of a consensus PPAR response element (3×PPRE), together with constructs expressing PPARα, RXRα. FIG. 8B shows luciferase expression in human embryonic kidney 293T (HEK293T) cells from FIG. 8A transfected with pCMV control (pCMV), SIRT4-Flag (SIRT4) or H161A-SIRT4-Flag (SIRT4 Mut). FIG. 8C shows luciferase expression in H2.35 hepatoma cells transfected with pCMV control (pCMV), SIRT4-Flag (SIRT4) or H161A-SIRT4-Flag (SIRT4 Mut).

FIGS. 9A and 9B show the oxidation of [³H]palmitate (nmol [³H]palmitate/h/mg protein) as analyzed using SIRT4 (−/−) and SIRT4 (+/+) MEFs (FIG. 9A) or SIRT4 (−/−) and SIRT4 (+/+) primary hepatocytes (FIG. 9B). FIG. 9C shows the consumption of palmitate from culture medium in SIRT4 (−/−) and (+/+) primary hepatocytes.

FIG. 10A shows the triglyceride (TG) levels in livers (m/mg tissue) of SIRT4 KO and WT mice after overnight fast (n=6 per genotype). FIG. 10B shows the fatty acid composition of triglycerides in livers of SIRT4 KO and WT mice after overnight fast. Data represent mean±SEM (n=6 per genotype). FIG. 10C shows the non-esterified fatty acids levels (NEFA, μM) in plasma of male SIRT4 KO and WT mice on a normal chow diet, before (0 h) and after fasting (16 h and 24 h).

FIG. 11 shows the overnight weight loss experienced by SIRT4 WT and SIRT4 KO mice during an overnight fast.

FIG. 12A shows growth curves of SIRT4 KO and WT mice on a low fat diet up to 6 months of age (n=10-12 per genotype, data represent mean±SEM). FIG. 12B shows the body weight of SIRT4 KO and WT mice on a high fat diet (HFD, 60% fat, Research diets) and WT mice on a low fat diet (LFD, 10% fat, Research diets). FIG. 12C shows the relative weight gain of SIRT4 KO and WT mice on HFD and WT mice on a LFD. FIG. 12D shows the weekly food intake (g/g BW) in SIRT4 KO and WT mice on HFD and WT mice on a LFD.

FIG. 13A shows the starting body weights of SIRT4 KO and WT mice on a HFD and WT mice on a LFD. FIG. 13B shows the starting age of SIRT4 KO and WT mice on a HFD and WT mice on a LFD.

FIG. 14 shows the daily food intake (g/g body weight) of SIRT4 KO and WT mice on a HFD and WT mice on a LFD.

FIG. 15A shows the total fecal output (48 h) of SIRT4 KO and WT mice on a HFD. FIG. 15B shows the total fecal output per bodyweight (48 h) of SIRT4 KO and WT mice on a HFD.

FIGS. 16A and 16B show the plasma triglycerides in fed or fasted SIRT4 KO and WT mice on a HFD or LFD. FIGS. 16C and 16D show the plasma NEFA in fed or fasted SIRT4 KO and WT mice on a HFD or LFD diet.

FIGS. 17A and 17B show the blood glucose levels in fed or fasted SIRT4 KO and WT mice on a HFD and WT mice on a LFD. FIG. 17C shows the liver weights of SIRT4 KO and WT mice after 16 weeks on HFD or SIRT4 WT mice on a LFD. FIG. 17D shows the Epididymal white adipose tissue (WAT) weights of SIRT4 KO and WT mice after 16 weeks on a HFD or SIRT4 WT mice on a LFD. FIGS. 17E and 17F show the insulin levels in plasma of fed or fasted SIRT4 KO and WT mice on a HFD and WT mice on a LFD. FIG. 17G shows the percent weight loss of SIRT4 KO mice on a HFD compared to WT mice on a HFD and WT mice on a LFD.

FIG. 18A shows a glucose tolerance test (GTT) performed in SIRT4 KO and WT mice on a HFD or WT mice on a LFD. (n=6 per group). FIG. 18B shows the area under curve of GTTs from FIG. 18A.

FIG. 19 shows a Western blot analysis performed on livers of overnight-fasted SIRT4 KO and SIRT4 WT mice using antibodies directed against phosho-acetyl-CoA carboxylase (p-ACC), acetyl-CoA carboxylase (ACC), phosho-AMP-activated kinase (p-AMPK), AMP-activated kinase (AMPK), SIRT4 and actin.

FIG. 20A shows the ATP and ADP levels (nmol/mg tissue) as measured in acid-soluble fractions from livers of overnight-fasted SIRT4 WT and SIRT4 KO mice. FIG. 20B shows the ATP/ADP ratio in SIRT4 WT and SIRT4 KO livers, calculated from the results presented in FIG. 20A.

FIG. 21A shows the NAD as measured from livers of SIRT4 KO and SIRT4 WT mice (fasted, n=6-8). Each data point represents the NAD concentration (pmol NAD/mg tissue) of one animal. The line represents the mean NAD concentration. FIG. 21B shows the NADH as measured from livers of SIRT4 KO and SIRT4 WT mice (fasted, n=6-8). Each data point represents the NADH concentration (pmol NADH/mg tissue) in one animal. The line represents the mean NADH concentration. FIG. 21C shows the NAD/NADH ratio from SIRT4 KO and SIRT4 WT whole liver tissue lysates. Each data point represents the NAD/NADH ratio in one animal. The line represents the mean NAD/NADH ratio.

FIG. 22 shows a western blot depicting the expression of SIRT1 and actin in whole liver lysates from fasted SIRT4 KO and WT mice.

FIG. 23 shows the oxidation of [³H]palmitate (nmol [³H]palmitate/h/mg protein) as analyzed using SIRT4 WT and SIRT4 KO primary hepatocytes either untreated, treated with the SIRT1 inhibitor Ex 527, or treated with etomoxir (ETO).

### DETAILED DESCRIPTION OF THE INVENTION

The embodiments and practices of the present invention, other embodiments, and their features and characteristics, will be apparent from the description, figures and claims that follow, with all of the claims hereby being incorporated by this reference into this Summary.

### DEFINITIONS

For convenience, certain terms employed in the specification, examples, and appended claims are collected here. Unless defined otherwise, all technical and scientific terms used herein have the same meaning as commonly understood by one of ordinary skill in the art to which this invention belongs.

The articles “a” and “an” are used herein to refer to one or to more than one (i.e., to at least one) of the grammatical object of the article. By way of example, “an element” means one element or more than one element.

The terms “test compound” and “agent” are used herein to denote a chemical compound, a small molecule, a mixture of chemical compounds, a biological macromolecule (such as a nucleic acid, an antibody, a protein or portion thereof, e.g., a peptide), or an extract made from biological materials such as bacteria, plants, fungi, or animal (particularly mammalian) cells or tissues. Test compounds and agents may be identified as having a particular activity by screening assays described herein below. The activity of such test compounds and agents may render them suitable as a “therapeutic compound” or a “therapeutic agent” which is a biologically, physiologically, or pharmacologically active substance (or substances) that acts locally or systemically in a subject. A test compound may be capable of and useful for binding to, agonizing, antagonizing, or otherwise modulating (regulating, modifying, upregulating, downregulating) the activity of a protein or complex of the invention.

The term “amino acid” is intended to embrace all molecules, whether natural or synthetic, which include both an amino functionality and an acid functionality and capable of being included in a polymer of naturally-occurring amino acids. Exemplary amino acids include naturally-occurring amino acids; analogs, derivatives and congeners thereof; amino acid analogs having variant side chains; and all stereoisomers of any of any of the foregoing.

The term “binding” or “interacting” refers to an association, which may be a stable association, between two molecules, e.g., between a polypeptide and a binding partner or agent, e.g., small molecule, due to, for example, electrostatic, hydrophobic, ionic and/or hydrogen-bond interactions under physiological conditions.

The terms “calorie restricted” and “calorie restriction” include any diet or feeding program to a mammal or other organism below ad libitum levels, such as 10%, 20%, 30%, 40%, 50% or more than 50% below ad libitum levels.

The term “chemical entity,” as used herein, refers to chemical compounds, complexes of two or more chemical compounds, and fragments of such compounds or complexes. In certain instances, it is desirable to use chemical entities exhibiting a wide range of structural and functional diversity, such as compounds exhibiting different shapes (e.g., flat aromatic rings(s), puckered aliphatic rings(s), straight and branched chain aliphatics with single, double, or triple bonds) and diverse functional groups (e.g., carboxylic acids, esters, ethers, amines, aldehydes, ketones, and various heterocyclic rings).

The term “complex” refers to an association between at least two moieties (e.g. chemical or biochemical) that have an affinity for one another. Examples of complexes include associations between antigen/antibodies, lectin/avidin, target polynucleotide/probe oligonucleotide, antibody/anti-antibody, receptor/ligand, enzyme/ligand, polypeptide/polypeptide, polypeptide/polynucleotide, polypeptide/co-factor, polypeptide/substrate, polypeptide/inhibitor, polypeptide/small molecule, and the like. “Member of a complex” refers to one moiety of the complex, such as a protein. “Protein complex” or “polypeptide complex” refers to a complex comprising at least two polypeptides or proteins.

The terms “comprise” and “comprising” are used in the inclusive, open sense, meaning that additional elements may be included.

When using the term “comprising” or “having” herein, it is understood that this term may also be replaced by the phrases “consisting essentially” of or “consisting of,” where appropriate. For example, “a fragment comprising amino acids 1-100 of sequence X” should be read as providing support for “a fragment consisting essentially of amino acids 1-100 of sequence X” as well as for “a fragment consisting of amino acids 1-100 of sequence X.”

The term “control” includes any portion of an experimental system designed to demonstrate that the factor being tested is responsible for the observed effect, and is therefore useful to isolate and quantify the effect of one variable on a system. A control includes a “reference sample” as described herein.

The term “druggable region”, when used in reference to a polypeptide, nucleic acid, complex and the like, refers to a region of the molecule which is a target or is a likely target for binding a modulator. For a polypeptide, a druggable region generally refers to a region wherein several amino acids of a polypeptide would be capable of interacting with a modulator or other molecule. For a polypeptide or complex thereof, exemplary druggable regions include binding pockets and sites, enzymatic active sites, interfaces between domains of a polypeptide or complex, surface grooves or contours or surfaces of a polypeptide or complex which are capable of participating in interactions with another molecule. In certain instances, the interacting molecule is another polypeptide, which may be naturally-occurring. A druggable region may be on the surface of the molecule.

Druggable regions may be described and characterized in a number of ways. For example, a druggable region may be characterized by some or all of the amino acids that make up the region, or the backbone atoms thereof, or the side chain atoms thereof (optionally with or without the Cα atoms). Alternatively, in certain instances, the volume of a druggable region corresponds to that of a carbon based molecule of at least about 200 amu and often up to about 800 amu. In other instances, it will be appreciated that the volume of such region may correspond to a molecule of at least about 600 amu and often up to about 1600 amu or more. Alternatively, a druggable region may be characterized by comparison to other regions on the same or other molecules. For example, the term “affinity region” refers to a druggable region on a molecule (such as a polypeptide of the invention) that is present in several other molecules, in so much as the structures of the same affinity regions are sufficiently the same so that they are expected to bind the same or related structural analogs. An example of an affinity region is an ATP-binding site of a protein kinase that is found in several protein kinases (whether or not of the same origin).

The term “selectivity region” refers to a druggable region of a molecule that may not be found on other molecules, in so much as the structures of different selectivity regions are sufficiently different so that they are not expected to bind the same or related structural analogs. An exemplary selectivity region is a catalytic domain of a protein kinase that exhibits specificity for one substrate. In certain instances, a single modulator may bind to the same affinity region across a number of proteins that have a substantially similar biological function, whereas the same modulator may bind to only one selectivity region of one of those proteins.

When used in reference to a druggable region, the “selectivity” or “specificity’ of a molecule such as a modulator to a druggable region may be used to describe the binding between the molecule and a druggable region. For example, the selectivity of a modulator with respect to a druggable region may be expressed by comparison to another modulator, using the respective values of Kd (i.e., the dissociation constants for each modulator-druggable region complex) or, in cases where a biological effect is observed below the Kd, the ratio of the respective EC50's (i.e., the concentrations that produce 50% of the maximum response for the modulator interacting with each druggable region).

A “form that is naturally occurring” when referring to a compound means a compound that is in a form, e.g., a composition, in which it can be found naturally. A compound is not in a form that is naturally occurring if, e.g., the compound has been purified and separated from at least some of the other molecules that are found with the compound in nature.

The term “isolated polypeptide” refers to a polypeptide, in certain embodiments prepared from recombinant DNA or RNA, or of synthetic origin, or some combination thereof, which (1) is not associated with proteins that it is normally found with in nature, (2) is isolated from the cell in which it normally occurs, (3) is isolated free of other proteins from the same cellular source, (4) is expressed by a cell from a different species, or (5) does not occur in nature.

The term “isolated nucleic acid” refers to a polynucleotide of genomic, cDNA, or synthetic origin or some combination there of, which (1) is not associated with the cell in which the “isolated nucleic acid” is found in nature, or (2) is operably linked to a polynucleotide to which it is not linked in nature.

The terms “label” or “labeled” refer to incorporation or attachment, optionally covalently or non-covalently, of a detectable marker into a molecule, such as a polypeptide.

The term “percent identical” refers to sequence identity between two amino acid sequences or between two nucleotide sequences. Identity can each be determined by comparing a position in each sequence which may be aligned for purposes of comparison. When an equivalent position in the compared sequences is occupied by the same base or amino acid, then the molecules are identical at that position; when the equivalent site occupied by the same or a similar amino acid residue (e.g., similar in steric and/or electronic nature), then the molecules can be referred to as homologous (similar) at that position. Expression as a percentage of homology, similarity, or identity refers to a function of the number of identical or similar amino acids at positions shared by the compared sequences. Expression as a percentage of homology, similarity, or identity refers to a function of the number of identical or similar amino acids at positions shared by the compared sequences. Various alignment algorithms and/or programs may be used, including FASTA, BLAST, or ENTREZ. FASTA and BLAST are available as a part of the GCG sequence analysis package (University of Wisconsin, Madison, Wis.), and can be used with, e.g., default settings. ENTREZ is available through the National Center for Biotechnology Information, National Library of Medicine, National Institutes of Health, Bethesda, Md. In one embodiment, the percent identity of two sequences can be determined by the GCG program with a gap weight of 1, e.g., each amino acid gap is weighted as if it were a single amino acid or nucleotide mismatch between the two sequences.

Other techniques for alignment are described in Methods in Enzymology, vol. 266: Computer Methods for Macromolecular Sequence Analysis (1996), ed. Doolittle, Academic Press, Inc., a division of Harcourt Brace & Co., San Diego, Calif., USA. Preferably, an alignment program that permits gaps in the sequence is utilized to align the sequences. The Smith-Waterman is one type of algorithm that permits gaps in sequence alignments. See Meth. Mol. Biol. 70: 173-187 (1997). Also, the GAP program using the Needleman and Wunsch alignment method can be utilized to align sequences. An alternative search strategy uses MPSRCH software, which runs on a MASPAR computer. MPSRCH uses a Smith-Waterman algorithm to score sequences on a massively parallel computer. This approach improves ability to pick up distantly related matches, and is especially tolerant of small gaps and nucleotide sequence errors. Nucleic acid-encoded amino acid sequences can be used to search both protein and DNA databases.

The term “mammal” is known in the art, and exemplary mammals include humans, primates, bovines, porcines, canines, felines, and rodents (e.g., mice and rats).

The term “modulation”, when used in reference to a functional property or biological activity or process (e.g., enzyme activity or receptor binding), refers to the capacity to either up regulate (e.g., activate or stimulate), down regulate (e.g., inhibit or suppress) or otherwise change a quality of such property, activity or process. In certain instances, such regulation may be contingent on the occurrence of a specific event, such as activation of a signal transduction pathway, and/or may be manifest only in particular cell types.

A “modulator” may be a polypeptide, nucleic acid, macromolecule, complex, molecule, small molecule, compound, species or the like (naturally-occurring or non-naturally-occurring), or an extract made from biological materials such as bacteria, plants, fungi, or animal cells or tissues, that may be capable of causing modulation. Modulators may be evaluated for potential activity as inhibitors or activators (directly or indirectly) of a functional property, biological activity or process, or combination of them, (e.g., agonist, partial antagonist, partial agonist, inverse agonist, antagonist, anti-microbial agents, inhibitors of microbial infection or proliferation, and the like) by inclusion in assays. In such assays, many modulators may be screened at one time. The activity of a modulator may be known, unknown or partially known.

The terms “polynucleotide”, and “nucleic acid” are used interchangeably. They refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified, such as by conjugation with a labeling component. The term “recombinant” polynucleotide means a polynucleotide of genomic, cDNA, semisynthetic, or synthetic origin which either does not occur in nature or is linked to another polynucleotide in a non-natural arrangement.

A “patient”, “subject” or “host” refers to either a human or a non-human animal.

The term “pharmaceutically acceptable carrier” is art-recognized and refers to a pharmaceutically-acceptable material, composition or vehicle, such as a liquid or solid filler, diluent, excipient, solvent or encapsulating material, involved in carrying or transporting any subject composition or component thereof from one organ, or portion of the body, to another organ, or portion of the body. Each carrier must be “acceptable” in the sense of being compatible with the subject composition and its components and not injurious to the patient. Some examples of materials which may serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The term “pharmaceutically-acceptable salts” is art-recognized and refers to the relatively non-toxic, inorganic and organic acid addition salts of compounds, including, for example, those contained in compositions described herein.

The terms “polypeptide fragment” or “fragment”, when used in reference to a reference polypeptide, refers to a polypeptide in which amino acid residues are deleted as compared to the reference polypeptide itself, but where the remaining amino acid sequence is usually identical to the corresponding positions in the reference polypeptide. Such deletions may occur at the amino-terminus or carboxy-terminus of the reference polypeptide, or alternatively both. Fragments typically are at least 5, 6, 8 or 10 amino acids long, at least 14 amino acids long, at least 20, 30, 40 or 50 amino acids long, at least 75 amino acids long, or at least 100, 150, 200, 300, 500 or more amino acids long. A fragment can retain one or more of the biological activities of the reference polypeptide. In certain embodiments, a fragment may comprise a druggable region, and optionally additional amino acids on one or both sides of the druggable region, which additional amino acids may number from 5, 10, 15, 20, 30, 40, 50, or up to 100 or more residues. Further, fragments can include a sub-fragment of a specific region, which sub-fragment retains a function of the region from which it is derived. In another embodiment, a fragment may have immunogenic properties. Fragments may be devoid of about 1, 2, 5, 10, 20, 50, 100 or more amino acids at the N- or C-terminus of the wildtype protein.

The term “small molecule” is art-recognized and refers to a composition which has a molecular weight of less than about 2000 amu, or less than about 1000 amu, and even less than about 500 amu. Small molecules may be, for example, nucleic acids, peptides, polypeptides, peptide nucleic acids, peptidomimetics, carbohydrates, lipids or other organic (carbon containing) or inorganic molecules. Many pharmaceutical companies have extensive libraries of chemical and/or biological mixtures, often fungal, bacterial, or algal extracts, which can be screened with any of the assays described herein. The term “small organic molecule” refers to a small molecule that is often identified as being an organic or medicinal compound, and does not include molecules that are exclusively nucleic acids, peptides or polypeptides.

A “sub-cellular fraction” is any portion of a cell or extra-cellular matrix, as produced by any fractionation or other method known in the art.

The term “substantially homologous,” when used in connection with amino acid sequences, refers to sequences which are substantially identical to or similar in sequence with each other, giving rise to a homology of conformation and thus to retention, to a useful degree, of one or more biological (including immunological) activities. The term is not intended to imply a common evolution of the sequences.

“Substantially purified” refers to a protein that has been separated from components which naturally accompany it. Preferably the protein is at least about 80%, more preferably at least about 90%, and most preferably at least about 99% of the total material (by volume, by wet or dry weight, or by mole percent or mole fraction) in a sample. Purity can be measured by any appropriate method, e.g., in the case of polypeptides by column chromatography, gel electrophoresis or HPLC analysis.

A “target protein” is any protein, peptide, or homolog thereof that is capable of being acted upon by a protein having an enzymatic or other activity, such as the activity of a SIRT4 protein.

A “target mRNA” is any messenger RNA transcript that is capable of being acted upon by an antagonistic nucleic acid that reduces expression or levels of the protein encoded by the mRNA.

### SIRT4 Proteins

As used herein, the term “SIRT4” or “SIRT4 protein” refers to proteins, e.g., eukaryotic proteins, e.g., mammalian proteins, comprising a mitochondrial protein having ADP-ribosyl transfer case activity, as well as functional domains, fragments (e.g., functional fragments), e.g., fragments of at least 8 amino acids, e.g., at least 8, 18, 28, 64, 128, 150, 180, 200, 220, 240, 260, or 280 amino acids, and variants thereof. Exemplary functional fragments of SIRT4 can, for example, have ADP-ribosyltransferase activity and/or the ability to interact with a SIRT4 binding partner. Exemplary SIRT4 proteins include those designated GenBank NM_{—}012240 (human SIRT4; SEQ ID NO: 1) and XM_{—}485674 (mouse SIRT4; SEQ ID NO: 2). Homologs of SIRT4 proteins will share 60%, 80%, 85%, 90%, 95%, 98%, 99% sequence identity to a known SIRT4 protein and feature an SIRT4 activity, e.g., ADP ribosylation, inhibition of fatty acid oxidation, and/or downregulation of glutamate dehydrogenase. Eukaryotic SIRT4 proteins may be localized, e.g., to mitochondria. Variants of SIRT4 proteins can be produced by standard means, including site-directed and random mutagenesis.

### Exemplary Compositions

Compositions comprising an isolated polypeptide or protein described herein, or a homolog thereof or may comprise less than about 25%, 10%, or alternatively about 5%, or alternatively about 1%, contaminating biological macromolecules or polypeptides. In certain embodiments, a composition contains a SIRT4 protein. Optionally, a composition contains a SIRT4 protein and a SIRT4-interacting protein. In other embodiments, the SIRT4 protein is a variant, such as H161YSIRT4.

In certain embodiments, a protein described herein is further linked to a heterologous polypeptide, e.g., a polypeptide comprising a domain which increases its solubility and/or facilitates its purification, identification, detection, and/or structural characterization. Exemplary domains, include, for example, glutathione S-transferase (GST), protein A, protein G, calmodulin-binding peptide, thioredoxin, maltose binding protein, HA, myc, poly arginine, poly His, poly His-Asp or FLAG fusion proteins and tags. Additional exemplary domains include domains that alter protein localization in vivo, such as signal peptides, type III secretion system-targeting peptides, transcytosis domains, nuclear localization signals, etc.

A protein described herein may be linked to at least 2, 3, 4, 5, or more heterologous polypeptides. Polypeptides may be linked to multiple copies of the same heterologous polypeptide or may be linked to two or more heterologous polypeptides. The fusions may occur at the N-terminus of the polypeptide, at the C-terminus of the polypeptide, or at both the N- and C-terminus of the polypeptide. It is also within the scope of the invention to include linker sequences between a protein described herein and the fusion domain in order to facilitate construction of the fusion protein or to optimize protein expression or structural constraints of the fusion protein. A polypeptide may also be constructed so as to contain protease cleavage sites between the fusion polypeptide and polypeptide of the invention in order to remove the tag after protein expression or thereafter. Examples of suitable endoproteases include, for example, Factor Xa and TEV proteases.

In another embodiment, a protein may be modified so that its rate of traversing the cellular membrane is increased. For example, the polypeptide may be fused to a second peptide which promotes “transcytosis,” e.g., uptake of the peptide by cells. The peptide may be a portion of the HIV transactivator (TAT) protein, such as the fragment corresponding to residues 37-62 or 48-60 of TAT, portions which have been observed to be rapidly taken up by a cell in vitro (Green and Loewenstein, (1989) Cell 55:1179-1188). Alternatively, the internalizing peptide may be derived from the Drosophila antennapedia protein, or homologs thereof. The 60 amino acid long homeodomain of the homeo-protein antennapedia has been demonstrated to translocate through biological membranes and can facilitate the translocation of heterologous polypeptides to which it is coupled. Thus, the polypeptide may be fused to a peptide consisting of about amino acids 42-58 of Drosophila antennapedia or shorter fragments for transcytosis (Derossi et al. (1996) J Biol Chem 271:18188-18193; Derossi et al. (1994) J Biol Chem 269:10444-10450; and Perez et al. (1992) J Cell Sci 102:717-722). The transcytosis polypeptide may also be a non-naturally-occurring membrane-translocating sequence (MTS), such as the peptide sequences disclosed in U.S. Pat. No. 6,248,558.

In another embodiment, a protein described herein is labeled with an isotopic label to facilitate its detection and or structural characterization using nuclear magnetic resonance or another applicable technique. Exemplary isotopic labels include radioisotopic labels such as, for example, potassium-40 (⁴⁰K), carbon-14 (¹⁴C), tritium (³H), sulphur-35 (³⁵S), phosphorus-32 (³²P), technetium-99m (^{99m}Tc), thallium-²⁰¹Tl), gallium-67 (⁶⁷Ga), indium-111 (¹¹¹In), iodine-123 (¹²³4 iodine-131 (¹³¹4 yttrium-90 (⁹⁰Y), samarium-153 (¹⁵³Sm), rhenium-186 (¹⁸⁶Re), rhenium-188 (¹⁸⁸Re), dysprosium-165 (¹⁶⁵Dy) and holmium-166 (¹⁶⁶Ho). The isotopic label may also be an atom with non zero nuclear spin, including, for example, hydrogen-1 (¹H), hydrogen-2 (²H), hydrogen-3 (³H), phosphorous-31 (³¹P), sodium-23 (²³Na), nitrogen-14 (¹⁴N), nitrogen-15 (¹⁵N), carbon-13 (¹³C) and fluorine-19 (¹⁹F). In certain embodiments, the polypeptide is uniformly labeled with an isotopic label, for example, wherein at least 50%, 70%, 80%, 90%, 95%, or 98% of the possible labels in the polypeptide are labeled, e.g., wherein at least 50%, 70%, 80%, 90%, 95%, or 98% of the nitrogen atoms in the polypeptide are ¹⁵N, and/or wherein at least 50%, 70%, 80%, 90%, 95%, or 98% of the carbon atoms in the polypeptide are ¹³C, and/or wherein at least 50%, 70%, 80%, 90%, 95%, or 98% of the hydrogen atoms in the polypeptide are ²H. In other embodiments, the isotopic label is located in one or more specific locations within the polypeptide, for example, the label may be specifically incorporated into one or more of the leucine residues of the polypeptide. The invention also encompasses the embodiment wherein a single polypeptide comprises two, three or more different isotopic labels; for example, the polypeptide comprises both ¹⁵N and ¹³C labeling.

In yet another embodiment, a protein described herein is labeled to facilitate structural characterization using x-ray crystallography or another applicable technique. Exemplary labels include heavy atom labels such as, for example, cobalt, selenium, krypton, bromine, strontium, molybdenum, ruthenium, rhodium, palladium, silver, cadmium, tin, iodine, xenon, barium, lanthanum, cerium, praseodymium, neodymium, samarium, europium, gadolinium, terbium, dysprosium, holmium, erbium, thulium, ytterbium, lutetium, tantalum, tungsten, rhenium, osmium, iridium, platinum, gold, mercury, thallium, lead, thorium and uranium. In an exemplary embodiment, the polypeptide is labeled with seleno-methionine.

A variety of methods are available for preparing a polypeptide with a label, such as a radioisotopic label or heavy atom label. For example, in one such method, an expression vector comprising a nucleic acid encoding a polypeptide is introduced into a host cell, and the host cell is cultured in a cell culture medium in the presence of a source of the label, thereby generating a labeled polypeptide. The extent to which a polypeptide may be labeled may vary.

In still another embodiment, a protein described herein is labeled with a fluorescent label to facilitate its detection, purification, or structural characterization. In an exemplary embodiment, the polypeptide of the invention is fused to a heterologous polypeptide sequence which produces a detectable fluorescent signal, including, for example, green fluorescent protein (GFP), enhanced green fluorescent protein (EGFP), Renilla Reniformis green fluorescent protein, GFPmut2, GFPuv4, enhanced yellow fluorescent protein (EYFP), enhanced cyan fluorescent protein (ECFP), enhanced blue fluorescent protein (EBFP), citrine and red fluorescent protein from discosoma (dsRED).

In other embodiments, a protein described herein is immobilized onto a solid surface, including, microtiter plates, slides, beads, films, etc. A protein described herein may be immobilized onto a “chip” as part of an array. An array, having a plurality of addresses, may comprise one or more polypeptides in one or more of those addresses.

In other embodiments, proteins described herein are contained within vessels useful for the manipulation of the polypeptide sample. For example, the polypeptide of the invention may be contained within a microtiter plate to facilitate detection, screening or purification of the polypeptide. The polypeptide may also be contained within a syringe as a container suitable for administering the polypeptide to a subject in order to generate antibodies or as part of a vaccination regimen. The polypeptides may also be contained within an NMR tube in order to enable characterization by nuclear magnetic resonance techniques.

In still other embodiments, the invention relates to a crystallized polypeptide of the invention and crystallized polypeptides which have been mounted for examination by x-ray crystallography as described further below. In certain instances, a protein described herein in crystal form may be single crystals of various dimensions (e.g., micro-crystals) or may be an aggregate of crystalline material.

In certain embodiments, it may be advantageous to provide naturally-occurring or experimentally-derived homologs of the polypeptide of the invention. Such homologs may function in as a modulator to promote or inhibit a subset of the biological activities of the naturally-occurring form of the polypeptide. Thus, specific biological effects may be elicited by treatment with a homolog of limited function, and with fewer side effects relative to treatment with agonists or antagonists which are directed to all of the biological activities of the polypeptide of the invention. For instance, antagonistic homologs may be generated which interfere with the ability of the wild-type polypeptide of the invention to associate with certain proteins, but which do not substantially interfere with the formation of complexes between the native polypeptide and other cellular proteins.

Nucleic acids encoding any of the proteins or homologs described herein are also provided herein. A nucleic acid may further be linked to a promoter and/or other regulatory sequences, as further described herein. Exemplary nucleic acids are those that are at least about 80%, 85%, 90%, 95%, 98%, 99% or 100% identical to a nucleotide sequence provided herein or a fragment thereof, such as nucleic acid sequence encoding the protein fragments described herein. Nucleic acids may also hybridize specifically, e.g., under stringent hybridization conditions, to a nucleic acid described herein or a fragment thereof.

Also provided herein are molecular complexes, e.g., protein complexes, comprising a SIRT4 protein or homolog thereof and a mitochondrial protein, and optionally other cofactors or molecules. Such compositions and complexes may be used, e.g., in screening assays to identify agents that modulate the interaction between a SIRT4 protein and a mitochondrial protein, and the interaction between an ADP ribosyl transferable and target protein.

Proteins and complexes described herein may exist in solution. A solution may be a composition, e.g., pharmaceutical composition, such as comprising a therapeutically acceptable diluent.

Proteins or complexes described herein may also exist in crystal form. A crystallized complex may include a protein described herein and one or more of the following: a histone or homolog thereof, a co-factor (such as a salt, metal, nucleotide, oligonucleotide or polypeptide), a modulator, or a small molecule. In another aspect, the present invention contemplates a crystallized complex including a polypeptide of the invention and any other molecule or atom (such as a metal ion) that associates with the polypeptide in vivo.

Also provided herein are antibodies that bind specifically to a complex between a SIRT4 protein or homolog thereof and a mitochondrial protein or homolog thereof, but essentially do not bind specifically to the SIRT4 protein or homolog alone nor to the mitochondrial protein or homolog alone. Also provided are antibodies that bind specifically to proteins or other biological molecules that are acted on by SIRT4.

Antibodies may be full length antibodies, fragments of antibodies (e.g., Fab or F(ab′)2), monoclonal antibodies, polyclonal antibodies, single chain antibodies, chimeric antibodies, humanized antibodies, human antibodies, mini antibodies or any other form of a molecule or complex of molecules that binds specifically to a molecular complex described herein.

### Screening Methods

Provided herein are screening methods for evaluating SIRT4 activity and for identifying test compounds or agents that modulate a SIRT4 activity, such as a fatty acid oxidation activity.

For example, the invention provides in part a method of evaluating SIRT4 fatty acid oxidation repression activity, the method comprising: providing a cell-free composition comprising a SIRT4 protein, an enzyme that catalyzes fatty acid oxidation, and a substrate, such as a fatty acid; and evaluating fatty acid oxidation activity in the composition. Preferably, the method additionally includes the step of including a test compound in the cell-free composition. The test compound may have an inhibitory property towards a SIRT4 protein, and the invention provides a method including the steps of contacting the SIRT4 protein with the test compound in the presence of an enzyme that catalyzes fatty acid oxidation, and a substrate, measuring the test rate of fatty acid oxidation in the presence of the test compound, and comparing the test rate of fatty acid oxidation with a control rate of fatty acid oxidation obtained in the absence of the test compound, wherein an increase in the test rate relative to the control rate is indicative of an inhibitory property of the test compound.

Alternatively, the test compound has a stimulatory property towards a SIRT4 protein, and the invention provides a method including the steps of contacting the SIRT4 protein with the test compound in the presence of an enzyme that catalyzes fatty acid oxidation, and a substrate, measuring the test rate of fatty acid oxidation in the presence of the test compound, and comparing the test rate of fatty acid oxidation with a control rate of fatty acid oxidation obtained in the absence of the test compound, wherein a decrease in the test rate relative to the control rate is indicative of a stimulatory property of the test compound.

The effect of a test compound on SIRT4 is determined by providing a reaction mixture comprising SIRT4 and a test compound, and evaluating an activity of SIRT4. The methods described herein can be performed in a multiplex or high-throughput format such that a plurality of test compounds from a chemical library. The reaction mixture is provided in vitro, such as a eukaryotic cell, such as a hepatocyte, brown adipose cell, and/or a muscle cell. Alternatively, the reaction mixture is provided in vivo, such as in a mammalian subject.

Non-limiting examples of tissues from which a cellular composition is obtained include liver, muscle, and brown adipose tissue (BAT). The cell or cell lysate may be from a eukaryotic cell, e.g., a mammalian cell (such as a human cell), a yeast cell, a non-human primate cell, a bovine cell, an ovine cell, an equine cell, a porcine cell, a sheep cell, a bird (e.g., chicken or fowl) cell, a canine cell, a feline cell or a rodent (mouse or rat) cell. It can also be a non-mammalian cell, e.g., a fish cell. Yeast cells include S. cerevisiae and C. albicans. The cell may also be a prokaryotic cell, e.g., a bacterial cell. The cell may also be a single-celled microorganism, e.g., a protozoan. The cell may also be a metazoan cell, a plant cell or an insect cell.

The method may further include determining the effect of a test compound or agent on a biological activity, e.g., a biological activity of SIRT4 or a complex thereof.

In certain embodiments, the invention provides contacting a SIRT4 protein with a cellular composition containing a target molecule, such as a protein, fatty acid, nucleic acid or similar biological moiety, whether naturally or synthetically derived, and a test compound, which has an inhibitory property or a stimulatory property directly on SIRT4, or other components of the cellular composition that interact with SIRT4.

A screening assay may also comprise using a cell or cell lysate or portion thereof, containing a SIRT4 protein and a target molecule; contacting the cell or cell lysate or portion thereof with a test compound; and determining whether the interaction between the SIRT4 protein and the target molecule is affected by the presence of the test compound. The SIRT4 protein and target molecule may be, e.g., proteins that are encoded by a heterologous or exogenous nucleic acid, i.e., a nucleic acid that is not present in a naturally occurring cell.

### Test Compounds

A compound or test compound can be any chemical compound, for example, a macromolecule (e.g., a polypeptide, a protein complex, or a nucleic acid) or a small molecule (e.g., an amino acid, a nucleotide, an organic or inorganic compound). The test compound can have a formula weight of less than about 10 000 grams per mole, less than 5 000 grams per mole, less than 1000 grams per mole, or less than about 500 grams per mole. The test compound can be naturally occurring (e.g., an herb or a nature product), synthetic, or both. Examples of macromolecules are proteins, protein complexes, and glycoproteins, nucleic acids, e.g., DNA, RNA (e.g., double stranded RNA or RNAi) and PNA (peptide nucleic acid). Examples of small molecules are peptides, peptidomimetics (e.g., peptoids), amino acids, amino acid analogs, polynucleotides, polynucleotide analogs, nucleotides, nucleotide analogs, nucleosides, glycosidic compounds, organic or inorganic compounds e.g., heteroorganic or organometallic compounds. A test compound can be the only substance assayed by the method described herein. Alternatively, a collection of test compounds can be assayed either consecutively or concurrently by the methods described herein.

In one embodiment, high throughput screening methods involve providing a combinatorial chemical or peptide library containing a large number of potential therapeutic compounds (potential modulator or ligand compounds). Such “combinatorial chemical libraries” or “ligand libraries” are then screened in one or more assays, as described herein, to identify those library members (particular chemical species or subclasses) that display a desired characteristic activity. The compounds thus identified can serve as conventional “lead compounds” or can themselves be used as potential or actual therapeutics.

A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Preparation and screening of combinatorial chemical libraries is well known to those of skill in the art. Such combinatorial chemical libraries include, but are not limited to, peptide libraries (see, e.g., U.S. Pat. No. 5,010,175; Furka, Int. J. Pept. Prot. Res. 37:487-493 (1991) and Houghton et al., Nature 354:84-88 (1991)). Other chemistries for generating chemical diversity libraries can also be used. Such chemistries include, but are not limited to: peptoids (e.g., PCT Publication No. WO 91/19735), encoded peptides (e.g., PCT Publication No. WO 93/20242), random bio-oligomers (e.g., PCT Publication No. WO 92/00091), benzodiazepines (e.g., U.S. Pat. No. 5,288,514), diversomers such as hydantoins, benzodiazepines and dipeptides (Hobbs et al., Proc. Nat. Acad. Sci. USA 90:6909-6913 (1993)), vinylogous polypeptides (Hagihara et al., J Amer. Chem. Soc. 114:6568 (1992)), nonpeptidal peptidomimetics with glucose scaffolding (Hirschmann et al., J Amer. Chem. Soc. 114:9217-9218 (1992)), analogous organic syntheses of small compound libraries (Chen et al., J. Amer. Chem. Soc. 116:2661 (1994)), oligocarbamates (Cho et al., Science 261:1303 (1993)), and/or peptidyl phosphonates (Campbell et al., J Org. Chem. 59:658 (1994)), nucleic acid libraries (see Ausubel, Berger and Sambrook, all supra), peptide nucleic acid libraries (see, e.g., U.S. Pat. No. 5,539,083), antibody libraries (see, e.g., Vaughn et al., Nature Biotechnology, 14(3):309-314 (1996) and PCT/US96/10287), carbohydrate libraries (see, e.g., Liang et al., Science, 274:1520-1522 (1996) and U.S. Pat. No. 5,593,853), small organic molecule libraries (see, e.g., benzodiazepines, Baum C&EN, January 18, page 33 (1993); isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, U.S. Pat. No. 5,288,514, and the like). Additional examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and Gallop et al. (1994) J Med. Chem. 37:1233.

Some exemplary libraries are used to generate variants from a particular lead compound. One method includes generating a combinatorial library in which one or more functional groups of the lead compound are varied, e.g., by derivatization. Thus, the combinatorial library can include a class of compounds which have a common structural feature (e.g., framework).

Devices for the preparation of combinatorial libraries are commercially available (see, e.g., 357 MPS, 390 MPS, Advanced Chem Tech, Louisville Ky.; SYMPHONY™, Rainin, Woburn, Mass.; 433A Applied Biosystems, Foster City, Calif.; 9050 Plus, Millipore, Bedford, Mass.). In addition, numerous combinatorial libraries are themselves commercially available (see, e.g., ComGenex, Princeton, N.J.; Asinex, Moscow, RU, Tripos, Inc., St. Louis, Mo.; ChemStar, Ltd, Moscow, RU; 3D Pharmaceuticals, Exton, Pa.; Martek Biosciences, Columbia, Md.; etc.).

Test compounds can also be obtained from biological libraries; peptoid libraries (libraries of molecules having the functionalities of peptides, but with a novel, non-peptide backbone which are resistant to enzymatic degradation but which nevertheless remain bioactive; see, e.g., Zuckermann, R. N. et al. (1994) J Med. Chem. 37:2678-85); spatially addressable parallel solid phase or solution phase libraries; synthetic library methods requiring deconvolution; the “one-bead one-compound” library method; and synthetic library methods using affinity chromatography selection. The biological libraries include libraries of nucleic acids and libraries of proteins. Some nucleic acid libraries encode a diverse set of proteins (e.g., natural and artificial proteins; others provide, for example, functional RNA and DNA molecules such as nucleic acid aptamers or ribozymes. A peptoid library can be made to include structures similar to a peptide library. (See also Lam (1997) Anticancer Drug Des. 12:145). A library of proteins may be produced by an expression library or a display library (e.g., a phage display library).

Libraries of compounds may be presented in solution (e.g., Houghten (1992) Biotechniques 13:412-421), or on beads (Lam (1991) Nature 354:82-84), chips (Fodor (1993) Nature 364:555-556), bacteria (Ladner, U.S. Pat. No. 5,223,409), spores (Ladner U.S. Pat. No. 5,223,409), plasmids (Cull et al. (1992) Proc Natl Acad Sci USA 89:1865-1869) or on phage (Scott and Smith (1990) Science 249:386-390; Devlin (1990) Science 249:404-406; Cwirla et al. (1990) Proc. Natl. Acad. Sci. 87:6378-6382; Felici (1991) J. Mol. Biol. 222:301-310).

### Methods of Using SIRT4 Polypeptides and Nucleic Acids

Provided herein are methods of determining a SIRT4 activity, particularly with regard to fatty acids and mitochondrial proteins such as the oxidative phosphorylation complex proteins. Also provided herein are methods for modulating the expression of genes that are regulated by SIRT4.

Exemplary methods for determining a SIRT4 activity include contacting a cellular composition comprising a target molecule with a SIRT4 protein, and measuring oxidation levels of the target molecule, as described herein. The cellular composition includes a mammal, a mammalian cell, a cellular component or sub-cellular fraction. A cellular composition may contain a liver cell or a muscle cell, or a cellular component or sub-cellular fraction of a liver or muscle cell, or mixtures of same. Advantageously, the cellular composition is obtained from a mammal subjected to a physiological stress, such as a calorie-restricted diet, a high fat diet, exercise or a combination thereof.

SIRT4 polypeptides and nucleic acids are also useful in methods of determining mitochondrial function in a mammalian subject based on a determination of the oxidation state of a biological molecule (e.g., a protein, lipid, nucleic acid, carbohydrate, hormone, growth factor, cytokine, or combination thereof) in a biological sample of the mammalian subject. Optionally, the method includes the further step of comparing the oxidation state of the mitochondrial biological molecule in the biological sample with an oxidation state of the mitochondrial biological molecule in a control or a reference sample. In certain embodiments, the reference sample comprises a biological sample obtained from a mammalian subject subjected to a physiological stress or has a reduced number of functional SIRT4 gene copies. Physiological stress is a calorie-restricted diet, a high fat diet, exercise or a combination thereof. In other embodiments, the biological sample is obtained from a mammalian subject suffering from or at risk of developing a fatty acid oxidation disorder (FOD), such as obesity, Medium Chain Acyl-CoA Dehydrogenase (MCAD) Deficiency, Short Chain Acyl-CoA Dehydrogenase (SCAD) Deficiency, long-chain Acyl-CoA dehydrogenase (LCAD) deficiency, Carnitine Palmityltransferase Translocase I & II Deficiency, Carnitine acylcarnitine translocase deficiency, Very Long Chain Acyl-CoA Dehydrogenase (VLCAD) Deficiency, Glutaricaciduria II, EFT Deficiency HMG Carnitine Transport Defect (Primary Carnitine Deficiency), Long Chain 3-Hydroxyacyl-CoA Dehydrogenase (LCHAD) Deficiency, Trifunctional Protein (TFP) Deficiency, 2,4 Dienoyl-CoA Reductase Deficiency, 3-Hydroxy Acyl CoA Dehydrogenase Deficiency (HADH), Electron Transfer Flavoprotein (ETF) Dehydrogenase Deficiency, or 3-Hydroxy-3 Methylglutaryl-CoA (HMG) Lyase Deficiency. The biological sample comprises, e.g., liver, kidney, brown adipose tissue or muscle. In some embodiments, the method further includes the step of administering to the mammalian subject a SIRT4 modulator.

Nucleic acids, e.g., those encoding a protein of interest or functional homolog thereof, or a nucleic acid intended to inhibit the production of a protein of interest (e.g., siRNA or antisense RNA) can be delivered to cells, e.g., eukaryotic cells, in culture, to cells ex vivo, and to cells in vivo. The cells can be of any type including without limitation cancer cells, stem cells, neuronal cells, and non-neuronal cells. The delivery of nucleic acids can be by any technique known in the art including viral mediated gene transfer, liposome mediated gene transfer, direct injection into a target tissue, organ, or tumor, injection into vasculature which supplies a target tissue or organ.

Polynucleotides can be administered in any suitable formulations known in the art. These can be as virus particles, as naked DNA, in liposomes, in complexes with polymeric carriers, etc. Polynucleotides can be administered to the arteries which feed a tissue or tumor. They can also be administered to adjacent tissue, whether tumor or normal, which could express the demethylase protein.

Nucleic acids can be delivered in any desired vector. These include viral or non-viral vectors, including adenovirus vectors, adeno-associated virus vectors, retrovirus vectors, lentivirus vectors, and plasmid vectors. Exemplary types of viruses include HSV (herpes simplex virus), AAV (adeno associated virus), HIV (human immunodeficiency virus), BIV (bovine immunodeficiency virus), and MLV (murine leukemia virus). Nucleic acids can be administered in any desired format that provides sufficiently efficient delivery levels, including in virus particles, in liposomes, in nanoparticles, and complexed to polymers.

The nucleic acids encoding a protein or nucleic acid of interest may be in a plasmid or viral vector, or other vector as is known in the art. Such vectors are well known and any can be selected for a particular application. In one embodiment of the invention, the gene delivery vehicle comprises a promoter and a demethylase coding sequence. Preferred promoters are tissue-specific promoters and promoters which are activated by cellular proliferation, such as the thymidine kinase and thymidylate synthase promoters. Other preferred promoters include promoters which are activatable by infection with a virus, such as the α- and β-interferon promoters, and promoters which are activatable by a hormone, such as estrogen. Other promoters which can be used include the Moloney virus LTR, the CMV promoter, and the mouse albumin promoter. A promoter may be constitutive or inducible.

In another embodiment, naked polynucleotide molecules are used as gene delivery vehicles, as described in WO 90/11092 and U.S. Pat. No. 5,580,859. Such gene delivery vehicles can be either growth factor DNA or RNA and, in certain embodiments, are linked to killed adenovirus. Curiel et al., Hum. Gene. Ther. 3:147-154, 1992. Other vehicles which can optionally be used include DNA-ligand (Wu et al., J. Biol. Chem. 264:16985-16987, 1989), lipid-DNA combinations (Felgner et al., Proc. Natl. Acad. Sci. USA 84:7413 7417, 1989), liposomes (Wang et al., Proc. Natl. Acad. Sci. 84:7851-7855, 1987) and microprojectiles (Williams et al., Proc. Natl. Acad. Sci. 88:2726-2730, 1991).

A gene delivery vehicle can optionally comprise viral sequences such as a viral origin of replication or packaging signal. These viral sequences can be selected from viruses such as astrovirus, coronavirus, orthomyxovirus, papovavirus, paramyxovirus, parvovirus, picornavirus, poxvirus, retrovirus, togavirus or adenovirus. In a preferred embodiment, the growth factor gene delivery vehicle is a recombinant retroviral vector. Recombinant retroviruses and various uses thereof have been described in numerous references including, for example, Mann et al., Cell 33:153, 1983, Cane and Mulligan, Proc. Nat'l. Acad. Sci. USA 81:6349, 1984, Miller et al., Human Gene Therapy 1:5-14, 1990, U.S. Pat. Nos. 4,405,712, 4,861,719, and 4,980,289, and PCT Application Nos. WO 89/02,468, WO 89/05,349, and WO 90/02,806. Numerous retroviral gene delivery vehicles can be utilized in the present invention, including for example those described in EP 0,415,731; WO 90/07936; WO 94/03622; WO 93/25698; WO 93/25234; U.S. Pat. No. 5,219,740; WO 9311230; WO 9310218; Vile and Hart, Cancer Res. 53:3860-3864, 1993; Vile and Hart, Cancer Res. 53:962-967, 1993; Ram et al., Cancer Res. 53:83-88, 1993; Takamiya et al., J. Neurosci. Res. 33:493-503, 1992; Baba et al., J. Neurosurg. 79:729-735, 1993 (U.S. Pat. No. 4,777,127, GB 2,200,651, EP 0,345,242 and WO91/02805).

A polynucleotide of interest can also be combined with a condensing agent to form a gene delivery vehicle. The condensing agent may be a polycation, such as polylysine, polyarginine, polyornithine, protamine, spermine, spermidine, and putrescine. Many suitable methods for making such linkages are known in the art.

In an alternative embodiment, a polynucleotide of interest is associated with a liposome to form a gene delivery vehicle. Liposomes are small, lipid vesicles comprised of an aqueous compartment enclosed by a lipid bilayer, typically spherical or slightly elongated structures several hundred Angstroms in diameter. Under appropriate conditions, a liposome can fuse with the plasma membrane of a cell or with the membrane of an endocytic vesicle within a cell which has internalized the liposome, thereby releasing its contents into the cytoplasm. Prior to interaction with the surface of a cell, however, the liposome membrane acts as a relatively impermeable barrier which sequesters and protects its contents, for example, from degradative enzymes. Additionally, because a liposome is a synthetic structure, specially designed liposomes can be produced which incorporate desirable features. See Stryer, Biochemistry, pp. 236-240, 1975 (W. H. Freeman, San Francisco, Calif.); Szoka et al., Biochim. Biophys. Acta 600:1, 1980; Bayer et al., Biochim. Biophys. Acta. 550:464, 1979; Rivnay et al., Meth. Enzymol. 149:119, 1987; Wang et al., PROC. NATL. ACAD. SCI. U.S.A. 84: 7851, 1987, Plant et al., Anal. Biochem. 176:420, 1989, and U.S. Pat. No. 4,762,915. Liposomes can encapsulate a variety of nucleic acid molecules including DNA, RNA, plasmids, and expression constructs comprising growth factor polynucleotides such those disclosed in the present invention.

Liposomal preparations for use in the present invention include cationic (positively charged), anionic (negatively charged) and neutral preparations. Cationic liposomes have been shown to mediate intracellular delivery of plasmid DNA (Felgner et al., Proc. Natl. Acad. Sci. USA 84:7413-7416, 1987), mRNA (Malone et al., Proc. Natl. Acad. Sci. USA 86:6077-6081, 1989), and purified transcription factors (Debs et al., J. Biol. Chem. 265:10189-10192, 1990), in functional form. Cationic liposomes are readily available. For example, N[1-2,3-dioleyloxy)propyl]-N,N,N-triethylammonium (DOTMA) liposomes are available under the trademark Lipofectin, from GIBCO BRL, Grand Island, N.Y. See also Felgner et al., Proc. Natl. Acad. Sci. USA 91: 5148-5152.87, 1994. Other commercially available liposomes include Transfectace (DDAB/DOPE) and DOTAP/DOPE (Boerhinger). Other cationic liposomes can be prepared from readily available materials using techniques well known in the art. See, e.g., Szoka et al., Proc. Natl. Acad. Sci. USA 75:4194-4198, 1978; and WO 90/11092 for descriptions of the synthesis of DOTAP (1,2-bis(oleoyloxy)-3-(trimethylammonio)propane) liposomes.

Similarly, anionic and neutral liposomes are readily available, such as from Avanti Polar Lipids (Birmingham, Ala.), or can be easily prepared using readily available materials. Such materials include phosphatidyl choline, cholesterol, phosphatidyl ethanolamine, dioleoylphosphatidyl choline (DOPC), dioleoylphosphatidyl glycerol (DOPG), and dioleoylphoshatidyl ethanolamine (DOPE), among others. These materials can also be mixed with the DOTMA and DOTAP starting materials in appropriate ratios. Methods for making liposomes using these materials are well known in the art.

One or more proteins (e.g., a SIRT4 protein, or a protein that modulates SIRT4 activity) or nucleic acid (e.g., siRNA) of interest may be encoded by a single nucleic acid delivered. Alternatively, separate nucleic acids may encode different protein or nucleic acids of interest. Different species of nucleic acids may be in different forms; they may use different promoters or different vectors or different delivery vehicles. Similarly, the same protein or nucleic acid of interest may be used in a combination of different forms.

### Oligonucleotide Inhibitors of SIRT4

In certain embodiments of the present invention, oligonucleotide inhibitors of SIRT4 are used. Oligonucleotide inhibitors include, but are not limited to, antisense molecules, siRNA molecules, shRNA molecules, ribozymes and triplex molecules. Such molecules are known in the art and the skilled artisan would be able to create oligonucleotide inhibitors of SIRT4 using routine methods.

Antisense molecules, siRNA or shRNA molecules, ribozymes or triplex molecules may be contacted with a cell or administered to an organism. Alternatively, constructs encoding such molecules may be contacted with or introduced into a cell or organism. Antisense constructs, antisense oligonucleotides, RNA interference constructs or siRNA duplex RNA molecules can be used to interfere with expression of a protein of interest, e.g., SIRT4 protein. Typically at least 15, 17, 19, or 21 nucleotides of the complement of the mRNA sequence are sufficient for an antisense molecule. Typically at least 15, 19, 21, 22, or 23 nucleotides of a target sequence are sufficient for an RNA interference molecule. In some embodiments, an RNA interference molecule will have a 2 nucleotide 3′ overhang. If the RNA interference molecule is expressed in a cell from a construct, for example from a hairpin molecule or from an inverted repeat of the SIRT4 gene sequence, then the endogenous cellular machinery may create the overhangs. siRNA molecules can be prepared by chemical synthesis, in vitro transcription, or digestion of long dsRNA by Rnase III or Dicer. These can be introduced into cells by transfection, electroporation, intracellular infection or other methods known in the art. See, for example: Hannon, GJ, 2002, RNA Interference, Nature 418: 244-251; Bernstein E et al., 2002, The rest is silence. RNA 7: 1509-1521; Hutvagner G et al., RNAi: Nature abhors a double-strand. Cur. Open. Genetics & Development 12: 225-232; Brummelkamp, 2002, A system for stable expression of short interfering RNAs in mammalian cells. Science 296: 550-553; Lee N S, Dohjima T, Bauer G, Li H, Li M-J, Ehsani A, Salvaterra P, and Rossi J. (2002). Expression of small interfering RNAs targeted against HIV-1 rev transcripts in human cells. Nature Biotechnol. 20:500-505; Miyagishi M, and Taira K. (2002). U6-promoter-driven siRNAs with four uridine 3′ overhangs efficiently suppress targeted gene expression in mammalian cells. Nature Biotechnol. 20:497-500; Paddison P J, Caudy A A, Bernstein E, Hannon G J, and Conklin D S. (2002). Short hairpin RNAs (shRNAs) induce sequence-specific silencing in mammalian cells. Genes & Dev. 16:948-958; Paul C P, Good P D, Winer I, and Engelke D R. (2002). Effective expression of small interfering RNA in human cells. Nature Biotechnol. 20:505-508; Sui G, Soohoo C, Affar E-B, Gay F, Shi Y, Forrester W C, and Shi Y. (2002). A DNA vector-based RNAi technology to suppress gene expression in mammalian cells. Proc. Natl. Acad. Sci. USA 99(6):5515-5520; Yu J-Y, DeRuiter S L, and Turner D L. (2002). RNA interference by expression of short-interfering RNAs and hairpin RNAs in mammalian cells. Proc. Natl. Acad. Sci. USA 99(9):6047-6052, PCT publications WO2006/066048 and WO2009/029688, US published application US2009/0123426, each of which is incorporated by reference in its entirety.

Antisense or RNA interference molecules can be delivered in vitro to cells or in vivo. Typical delivery means known in the art can be used. Other modes of delivery can be used without limitation, including: intravenous, intramuscular, intraperitoneal, intraarterial, local delivery during surgery, endoscopic, subcutaneous, and per os. Vectors can be selected for desirable properties for any particular application. Vectors can be viral, bacterial or plasmid. Adenoviral vectors are useful in this regard. Tissue-specific, cell-type specific, or otherwise regulatable promoters can be used to control the transcription of the inhibitory polynucleotide molecules. Non-viral carriers such as liposomes or nanospheres can also be used.

In the present methods, a RNA interference molecule or an RNA interference encoding oligonucleotide can be administered to the subject, for example, as naked RNA, in combination with a delivery reagent, and/or as a nucleic acid comprising sequences that express the siRNA or shRNA molecules. In some embodiments the nucleic acid comprising sequences that express the siRNA or shRNA molecules are delivered within vectors, e.g. plasmid, viral and bacterial vectors. Any nucleic acid delivery method known in the art can be used in the present invention. Suitable delivery reagents include, but are not limited to, e.g., the Minis Transit TKO lipophilic reagent; lipofectin; lipofectamine; cellfectin; polycations (e.g., polylysine), atelocollagen, nanoplexes and liposomes.

The use of atelocollagen as a delivery vehicle for nucleic acid molecules is described in Minakuchi et al. Nucleic Acids Res., 32(13):e109 (2004); Hanai et al. Ann NY Acad. Sci., 1082:9-17 (2006); and Kawata et al. Mol Cancer Ther., 7(9):2904-12 (2008); each of which is incorporated herein in their entirety.

In some embodiments of the invention, liposomes are used to deliver an inhibitory oligonucleotide to a subject. Liposomes suitable for use in the invention can be formed from standard vesicle-forming lipids, which generally include neutral or negatively charged phospholipids and a sterol, such as cholesterol. The selection of lipids is generally guided by consideration of factors such as the desired liposome size and half-life of the liposomes in the blood stream. A variety of methods are known for preparing liposomes, for example, as described in Szoka et al. (1980), Ann. Rev. Biophys. Bioeng. 9:467; and U.S. Pat. Nos. 4,235,871, 4,501,728, 4,837,028, and 5,019,369, the entire disclosures of which are herein incorporated by reference.

The liposomes for use in the present methods can also be modified so as to avoid clearance by the mononuclear macrophage system (“MMS”) and reticuloendothelial system (“RES”). Such modified liposomes have opsonization-inhibition moieties on the surface or incorporated into the liposome structure. In an embodiment, a liposome of the invention can comprise both opsonization-inhibition moieties and a ligand.

Opsonization-inhibiting moieties for use in preparing the liposomes of the invention are typically large hydrophilic polymers that are bound to the liposome membrane. As used herein, an opsonization inhibiting moiety is “bound” to a liposome membrane when it is chemically or physically attached to the membrane, e.g., by the intercalation of a lipid-soluble anchor into the membrane itself, or by binding directly to active groups of membrane lipids. These opsonization-inhibiting hydrophilic polymers form a protective surface layer that significantly decreases the uptake of the liposomes by the MMS and RES; e.g., as described in U.S. Pat. No. 4,920,016, the entire disclosure of which is herein incorporated by reference.

Opsonization inhibiting moieties suitable for modifying liposomes are preferably water-soluble polymers with a number-average molecular weight from about 500 to about 40,000 daltons, and more preferably from about 2,000 to about 20,000 daltons. Such polymers include polyethylene glycol (PEG) or polypropylene glycol (PPG) derivatives; e.g., methoxy PEG or PPG, and PEG or PPG stearate; synthetic polymers such as polyacrylamide or poly N-vinyl pyrrolidone; linear, branched, or dendrimeric polyamidoamines; polyacrylic acids; polyalcohols, e.g., polyvinylalcohol and polyxylitol to which carboxylic or amino groups are chemically linked, as well as gangliosides, such as ganglioside GM1. Copolymers of PEG, methoxy PEG, or methoxy PPG, or derivatives thereof, are also suitable. In addition, the opsonization inhibiting polymer can be a block copolymer of PEG and either a polyamino acid, polysaccharide, polyamidoamine, polyethyleneamine, or polynucleotide. The opsonization inhibiting polymers can also be natural polysaccharides containing amino acids or carboxylic acids, e.g., galacturonic acid, glucuronic acid, mannuronic acid, hyaluronic acid, pectic acid, neuraminic acid, alginic acid, carrageenan; aminated polysaccharides or oligosaccharides (linear or branched); or carboxylated polysaccharides or oligosaccharides, e.g., reacted with derivatives of carbonic acids with resultant linking of carboxylic groups. Preferably, the opsonization-inhibiting moiety is a PEG, PPG, or derivatives thereof. Liposomes modified with PEG or PEG-derivatives are sometimes called “PEGylated liposomes.”

The opsonization inhibiting moiety can be bound to the liposome membrane by any one of numerous well-known techniques. For example, an N-hydroxysuccinimide ester of PEG can be bound to a phosphatidyl-ethanolamine lipid-soluble anchor, and then bound to a membrane. Similarly, a dextran polymer can be derivatized with a stearylamine lipid-soluble anchor via reductive amination using Na(CN)BH₃ and a solvent mixture, such as tetrahydrofuran and water in a 30:12 ratio at 60° C.

Liposomes modified with opsonization-inhibition moieties remain in the circulation much longer than unmodified liposomes. For this reason, such liposomes are sometimes called “stealth” liposomes. Stealth liposomes are known to accumulate in tissues fed by porous or “leaky” microvasculature. Thus, tissue characterized by such microvasculature defects, for example solid tumors, will efficiently accumulate these liposomes; see Gabizon, et al. (1988), Proc. Natl. Acad. Sci., USA, 18:6949-53. In addition, the reduced uptake by the RES lowers the toxicity of stealth liposomes by preventing significant accumulation of the liposomes in the liver and spleen.

### Antibody Inhibitors of SIRT4

Because of their ability to bind to a particular target with high specificity, antibodies specific for SIRT4 are able to inhibit SIRT4 activity. Though antibodies are most often used to inhibit the activity of extracellular proteins (e.g., receptors and/or ligands), the use of intracellular antibodies to inhibit protein function in a cell is also known in the art (see e.g., Carlson, J. R. (1988) Mol. Cell. Biol. 8:2638-2646; Biocca, S. et al. (1990) EMBO J. 9:101-108; Werge, T. M. et al. (1990) FEBS Lett. 274:193-198; Carlson, J. R. (1993) Proc. Natl. Acad. Sci. USA 90:7427-7428; Marasco, W. A. et al. (1993) Proc. Natl. Acad. Sci. USA 90:7889-7893; Biocca, S. et al. (1994) Biotechnology (NY) 12:396-399; Chen, S-Y. et al. (1994) Hum. Gene Ther. 5:595-601; Duan, L et al. (1994) Proc. Natl. Acad. Sci. USA 91:5075-5079; Chen, S-Y. et al. (1994) Proc. Natl. Acad. Sci. USA 91:5932-5936; Beerli, R. R. et al. (1994) J. Biol. Chem. 269:23931-23936; Beerli, R. R. et al. (1994) Biochem. Biophys. Res. Commun. 204:666-672; Mhashilkar, A. M. et al. (1995) EMBO J. 14:1542-1551; Richardson, J. H. et al. (1995) Proc. Natl. Acad. Sci. USA 92:3137-3141; PCT Publication No. WO 94/02610 by Marasco et al.; and PCT Publication No. WO 95/03832 by Duan et al.). Therefore, antibodies specific for SIRT4 are useful agents for the methods of the present invention.

Antibodies that specifically bind to SIRT4 can be produced using a variety of known techniques, such as the standard somatic cell hybridization technique described by Kohler and Milstein, Nature 256: 495 (1975). Additionally, other techniques for producing monoclonal antibodies known in the art can also be employed, e.g., viral or oncogenic transformation of B lymphocytes, phage display technique using libraries of human antibody genes.

Polyclonal antibodies can be prepared by immunizing a suitable subject with a polypeptide immunogen. The polypeptide antibody titer in the immunized subject can be monitored over time by standard techniques, such as with an enzyme linked immunosorbent assay (ELISA) using immobilized polypeptide. If desired, the antibody directed against the antigen can be isolated from the mammal (e.g., from the blood) and further purified by well known techniques, such as protein A chromatography to obtain the IgG fraction. At an appropriate time after immunization, e.g., when the antibody titers are highest, antibody-producing cells can be obtained from the subject and used to prepare monoclonal antibodies.

Any of the many well known protocols used for fusing lymphocytes and immortalized cell lines can be applied for the purpose of generating monoclonal antibodies specific against SIRT4 (see, e.g., Galfre, G. et al. (1977) Nature 266:55052; Gefter et al. (1977) supra; Lerner (1981) supra; Kenneth (1980) supra). Moreover, the ordinary skilled worker will appreciate that there are many variations of such methods which also would be useful. Typically, an immortal cell line (e.g., a myeloma cell line) is derived from the same mammalian species as the lymphocytes. For example, murine hybridomas can be made by fusing lymphocytes from a mouse immunized with an immunogenic preparation of the present invention with an immortalized mouse cell line. An example of an appropriate mouse cell lines are mouse myeloma cell lines that are sensitive to culture medium containing hypoxanthine, aminopterin and thymidine (“HAT medium”). Any of a number of myeloma cell lines can be used as a fusion partner according to standard techniques, e.g., the P3-NS1/1-Ag4-1, P3-x63-Ag8.653 or Sp2/O-Ag14 myeloma lines. These myeloma lines are available from the American Type Culture Collection (ATCC), Rockville, Md. Typically, HAT-sensitive mouse myeloma cells are fused to mouse splenocytes using polyethylene glycol (“PEG”). Hybridoma cells resulting from the fusion are then selected using HAT medium, which kills unfused and unproductively fused myeloma cells (unfused splenocytes die after several days because they are not transformed). Hybridoma cells producing a monoclonal antibody of the invention are detected by screening the hybridoma culture supernatants for antibodies that bind a given polypeptide, e.g., using a standard ELISA assay.

As an alternative to preparing monoclonal antibody-secreting hybridomas, a monoclonal antibody specific for SIRT4 can be identified and isolated by screening a recombinant combinatorial immunoglobulin library (e.g., an antibody phage or yeast display library) with the appropriate SIRT4 to thereby isolate immunoglobulin library members that bind SIRT4. Kits for generating and screening phage display libraries are commercially available (e.g., the Pharmacia Recombinant Phage Antibody System, Catalog No. 27-9400-01; and the Stratagene SurfZAP™ Phage Display Kit, Catalog No. 240612), and methods for screening phage and yeast display libraries are known in the art. Examples of methods and reagents particularly amenable for use in generating and screening an antibody display library can be found in, for example, Ladner et al. U.S. Pat. No. 5,223,409; Kang et al. International Publication No. WO 92/18619; Dower et al. International Publication No. WO 91/17271; Winter et al. International Publication WO 92/20791; Markland et al. International Publication No. WO 92/15679; Breitling et al. International Publication WO 93/01288; McCafferty et al. International Publication No. WO 92/01047; Garrard et al. International Publication No. WO 92/09690; Ladner et al. International Publication No. WO 90/02809; Fuchs et al. (1991) Biotechnology (NY) 9:1369-1372; Hay et al. (1992) Hum. Antibod. Hybridomas 3:81-85; Huse et al. (1989) Science 246:1275-1281; Griffiths et al. (1993) EMBO J. 12:725-734; Hawkins et al. (1992) J. Mol. Biol. 226:889-896; Clarkson et al. (1991) Nature 352:624-628; Gram et al. (1992) Proc. Natl. Acad. Sci. USA 89:3576-3580; Garrard et al. (1991) Biotechnology (NY) 9:1373-1377; Hoogenboom et al. (1991) Nucleic Acids Res. 19:4133-4137; Barbas et al. (1991) Proc. Natl. Acad. Sci. USA 88:7978-7982; and McCafferty et al. (1990) Nature 348:552-554.

In addition, chimeric and humanized antibodies against SIRT4 can be made according to standard protocols such as those disclosed in U.S. Pat. No. 5,565,332. In another embodiment, antibody chains or specific binding pair members can be produced by recombination between vectors comprising nucleic acid molecules encoding a fusion of a polypeptide chain of a specific binding pair member and a component of a replicable generic display package and vectors containing nucleic acid molecules encoding a second polypeptide chain of a single binding pair member using techniques known in the art, e.g., as described in U.S. Pat. Nos. 5,565,332, 5,871,907, or 5,733,743.

In another embodiment, human monoclonal antibodies directed against SIRT4 can be generated using transgenic or transchromosomal mice carrying parts of the human immune system rather than the mouse system. In one embodiment, transgenic mice, referred to herein as “humanized mice,” which contain a human immunoglobulin gene miniloci that encodes unrearranged human heavy and light chain variable region immunoglobulin sequences, together with targeted mutations that inactivate or delete the endogenous μ and κ chain loci (Lonberg, N. et al. (1994) Nature 368(6474): 856 859). The mice may also contain human heavy chain constant region immunoglobulin sequences. Accordingly, the mice express little or no mouse IgM or κ, and in response to immunization, the introduced human heavy and light chain variable region transgenes undergo class switching and somatic mutation to generate high affinity human variable region antibodies (Lonberg, N. et al. (1994), supra; reviewed in Lonberg, N. (1994) Handbook of Experimental Pharmacology 113:49 101; Lonberg, N. and Huszar, D. (1995) Intern. Rev. Immunol. Vol. 13: 65 93, and Harding, F. and Lonberg, N. (1995) Ann. N.Y. Acad. Sci. 764:536 546). These mice can be used to generate fully human monoclonal antibodies using the techniques described above or any other technique known in the art. The preparation of humanized mice is described in Taylor, L. et al. (1992) Nucleic Acids Research 20:6287 6295; Chen, J. et al. (1993) International Immunology 5: 647 656; Tuaillon et al. (1993) Proc. Natl. Acad. Sci. USA 90:3720 3724; Choi et al. (1993) Nature Genetics 4:117 123; Chen, J. et al. (1993) EMBO J. 12: 821 830; Tuaillon et al. (1994) J. Immunol. 152:2912 2920; Lonberg et al., (1994) Nature 368(6474): 856 859; Lonberg, N. (1994) Handbook of Experimental Pharmacology 113:49 101; Taylor, L. et al. (1994) International Immunology 6: 579 591; Lonberg, N. and Huszar, D. (1995) Intern. Rev. Immunol. Vol. 13: 65 93; Harding, F. and Lonberg, N. (1995) Ann. N.Y. Acad. Sci 764:536 546; Fishwild, D. et al. (1996) Nature Biotechnology 14: 845 851. See further, U.S. Pat. Nos. 5,545,806; 5,569,825; 5,625,126; 5,633,425; 5,789,650; 5,877,397; 5,661,016; 5,814,318; 5,874,299; and 5,770,429; all to Lonberg and Kay, and GenPharm International; U.S. Pat. No. 5,545,807 to Surani et al.

### Exemplary Methods of Treatment and Diseases

Provided herein are methods of treatment or prevention of conditions and diseases that can be improved by modulating the level or activity of SIRT4.

For example, provided herein are methods of treating or preventing a mitochondrial disease in a mammalian subject, comprising administering to the subject an effective amount of an agent that modulates SIRT4 protein activity. The mitochondrial disease is, e.g., a fatty acid oxidation disorder (FOD) such as obesity, Medium Chain Acyl-CoA Dehydrogenase (MCAD) Deficiency, Short Chain Acyl-CoA Dehydrogenase (SCAD) Deficiency, long-chain Acyl-CoA dehydrogenase (LCAD) deficiency, Carnitine Palmityltransferase Translocase I & II Deficiency, Carnitine acylcarnitine translocase deficiency, Very Long Chain Acyl-CoA Dehydrogenase (VLCAD) Deficiency, Glutaricaciduria II, EFT Deficiency HMG Carnitine Transport Defect (Primary Carnitine Deficiency), Long Chain 3-Hydroxyacyl-CoA Dehydrogenase (LCHAD) Deficiency, Trifunctional Protein (TFP) Deficiency, 2,4 Dienoyl-CoA Reductase Deficiency, 3-Hydroxy Acyl CoA Dehydrogenase Deficiency (HADH), Electron Transfer Flavoprotein (ETF) Dehydrogenase Deficiency, or 3-Hydroxy-3 Methylglutaryl-CoA (HMG) Lyase Deficiency. In some embodiments, the levels of SIRT4 are modulated in a brown adipose tissue, hepatocyte or a muscle cell. In other embodiments, the agent is an antagonistic nucleic acid that reduces SIRT4 expression. For example, the agent comprises a nucleic acid that targets SIRT4 mRNA or an antibody that targets SIRT4 protein.

In certain embodiments, the invention relates to methods of preventing diet-induced weight gain in a subject through the administration of an agent that reduces the level or activity of SIRT4. In some embodiments, the invention relates to methods of treating steatosis in a subject through the administration of an agent that reduces the level or activity of SIRT4 in the subject. In other embodiments, the invention relates to methods of treating lipodystrophies or other fatty acid storage diseases in a subject through the administration of an agent that increases the level of activity of SIRT4 in the subject.

Mitochondrial dysfunction is associated with the onset and progression of cancer. Exemplary cancers that may be treated include leukemias, e.g., acute lymphoid leukemia and myeloid leukemia, and carcinomas, such as colorectal carcinoma and hepatocarcinoma. Other cancers include Acute Lymphoblastic Leukemia; Acute Lymphoblastic Leukemia; Acute Myeloid Leukemia; Acute Myeloid Leukemia; Adrenocortical Carcinoma Adrenocortical Carcinoma; AIDS-Related Cancers; AIDS-Related Lymphoma; Anal Cancer; Astrocytoma, Childhood Cerebellar; Astrocytoma, Childhood Cerebral; Basal Cell Carcinoma, see Skin Cancer (non-Melanoma); Bile Duct Cancer, Extrahepatic; Bladder Cancer; Bladder Cancer; Bone Cancer, osteosarcoma/Malignant Fibrous Histiocytoma; Brain Stem Glioma; Brain Tumor; Brain Tumor, Brain Stem Glioma; Brain Tumor, Cerebellar Astrocytoma; Brain Tumor, Cerebral Astrocytoma/Malignant Glioma; Brain Tumor, Ependymoma; Brain Tumor, Medulloblastoma; Brain Tumor, Supratentorial Primitive Neuroectodermal Tumors; Brain Tumor, Visual Pathway and Hypothalamic Glioma; Brain Tumor; Breast Cancer; Breast Cancer and Pregnancy; Breast Cancer; Breast Cancer, Male; Bronchial Adenomas/Carcinoids; Burkitt's Lymphoma; Carcinoid Tumor; Carcinoid Tumor, Gastrointestinal; Carcinoma of Unknown Primary; Central Nervous System Lymphoma, Primary; Cerebellar Astrocytoma; Cerebral Astrocytoma/Malignant Glioma; Cervical Cancer; Childhood Cancers; Chronic Lymphocytic Leukemia; Chronic Myelogenous Leukemia; Chronic Myeloproliferative Disorders; Colon Cancer; Colorectal Cancer; Cutaneous T-Cell Lymphoma, see Mycosis Fungoides and Séezary Syndrome; Endometrial Cancer; Ependymoma; Esophageal Cancer; Esophageal Cancer; Ewing's Family of Tumors; Extracranial Germ Cell Tumor; Extragonadal Germ Cell Tumor; Extrahepatic Bile Duct Cancer; Eye Cancer, Intraocular Melanoma; Eye Cancer, Retinoblastoma; Gallbladder Cancer; Gastric (Stomach) Cancer; Gastric (Stomach) Cancer; Gastrointestinal Carcinoid Tumor; Germ Cell Tumor, Extracranial; Germ Cell Tumor, Extragonadal; Germ Cell Tumor, Ovarian; Gestational Trophoblastic Tumor; Glioma; Glioma, Childhood Brain Stem; Glioma, Childhood Cerebral Astrocytoma; Glioma, Childhood Visual Pathway and Hypothalamic; Hairy Cell Leukemia; Head and Neck Cancer; Hepatocellular (Liver) Cancer, Adult (Primary); Hepatocellular (Liver) Cancer, Childhood (Primary); Hodgkin's Lymphoma; Hodgkin's Lymphoma; Hodgkin's Lymphoma During Pregnancy; Hypopharyngeal Cancer; Hypothalamic and Visual Pathway Glioma; Intraocular Melanoma; Islet Cell Carcinoma (Endocrine Pancreas); Kaposi's Sarcoma; Kidney (Renal Cell) Cancer; Kidney Cancer; Laryngeal Cancer; Laryngeal Cancer; Leukemia, Acute Lymphoblastic; Leukemia, Acute Lymphoblastic; Leukemia, Acute Myeloid; Leukemia, Acute Myeloid; Leukemia, Chronic Lymphocytic; Leukemia; Chronic Myelogenous; Leukemia, Hairy Cell; Lip and Oral Cavity Cancer; Liver Cancer, Adult (Primary); Liver Cancer, Childhood (Primary); Lung Cancer, Non-Small Cell; Lung Cancer, Small Cell; Lymphoma, AIDS-Related; Lymphoma, Burkitt's; Lymphoma, Cutaneous T-Cell, see Mycosis Fungoides and Sézary Syndrome; Lymphoma, Hodgkin's; Lymphoma, Hodgkin's; Lymphoma, Hodgkin's During Pregnancy; Lymphoma, Non-Hodgkin's; Lymphoma, Non-Hodgkin's; Lymphoma, Non-Hodgkin's During Pregnancy; Lymphoma, Primary Central Nervous System; Macroglobulinemia, Waldenström's; Malignant Fibrous Histiocytoma of Bone/Osteosarcoma; Medulloblastoma; Melanoma; Melanoma, Intraocular (Eye); Merkel Cell Carcinoma; Mesothelioma, Adult Malignant; Mesothelioma; Metastatic Squamous Neck Cancer with Occult Primary; Multiple Endocrine Neoplasia Syndrome; Multiple Myeloma/Plasma Cell Neoplasm' Mycosis Fungoides; Myelodysplastic Syndromes; Myelodysplastic/Myeloproliferative Diseases; Myelogenous Leukemia, Chronic; Myeloid Leukemia, Adult Acute; Myeloid Leukemia, Childhood Acute; Myeloma, Multiple; Myeloproliferative Disorders, Chronic; Nasal Cavity and Paranasal Sinus Cancer; Nasopharyngeal Cancer; Nasopharyngeal Cancer; Neuroblastoma; Non-Hodgkin's Lymphoma;
Non-Hodgkin's Lymphoma; Non-Hodgkin's Lymphoma During Pregnancy; Non-Small Cell Lung Cancer; Oral Cancer; Oral Cavity Cancer, Lip and; Oropharyngeal Cancer; Osteosarcoma/Malignant Fibrous Histiocytoma of Bone; Ovarian Cancer; Ovarian Epithelial Cancer; Ovarian Germ Cell Tumor; Ovarian Low Malignant Potential Tumor; Pancreatic Cancer; Pancreatic Cancer; Pancreatic Cancer, Islet Cell; Paranasal Sinus and Nasal Cavity Cancer; Parathyroid Cancer; Penile Cancer; Pheochromocytoma; Pineoblastoma and Supratentorial Primitive Neuroectodermal Tumors; Pituitary Tumor; Plasma Cell Neoplasm/Multiple Myeloma; Pleuropulmonary Blastoma; Pregnancy and Breast Cancer; Pregnancy and Hodgkin's Lymphoma; Pregnancy and Non-Hodgkin's Lymphoma; Primary Central Nervous System Lymphoma; Prostate Cancer; Rectal Cancer; Renal Cell (Kidney) Cancer; Renal Cell (Kidney) Cancer; Renal Pelvis and Ureter, Transitional Cell Cancer; Retinoblastoma; Rhabdomyosarcoma; Salivary Gland Cancer; Salivary Gland Cancer; Sarcoma, Ewing's Family of Tumors; Sarcoma, Kaposi's; Sarcoma, Soft Tissue; Sarcoma, Soft Tissue; Sarcoma, Uterine; Sezary Syndrome; Skin Cancer (non-Melanoma); Skin Cancer; Skin Cancer (Melanoma); Skin Carcinoma, Merkel Cell; Small Cell Lung Cancer; Small Intestine Cancer; Soft Tissue Sarcoma; Soft Tissue Sarcoma; Squamous Cell Carcinoma, see Skin Cancer (non-Melanoma); Squamous Neck Cancer with Occult Primary, Metastatic; Stomach (Gastric) Cancer; Stomach (Gastric) Cancer; Supratentorial Primitive Neuroectodermal Tumors; T-Cell Lymphoma, Cutaneous, see Mycosis Fungoides and Sézary Syndrome; Testicular Cancer; Thymoma; Thymoma and Thymic Carcinoma; Thyroid Cancer; Thyroid Cancer; Transitional Cell Cancer of the Renal Pelvis and Ureter; Trophoblastic Tumor, Gestational; Unknown Primary Site, Carcinoma of; Unknown Primary Site, Cancer of; Unusual Cancers of Childhood; Ureter and Renal Pelvis, Transitional Cell Cancer; Urethral Cancer; Uterine Cancer, Endometrial; Uterine Sarcoma; Vaginal Cancer; Visual Pathway and Hypothalamic Glioma; Vulvar Cancer; Waldenström's Macroglobulinemia; Wilms' Tumor; and Women's Cancers.

Any other disease in which epigenetics factors play a role is likely to be treatable or preventable by applying methods described herein.

In some embodiments, the present invention relates to methods of inducing weight gain, fatty acid deposition or of treating lipodystrophy in a mammalian subject by administering to the subject an agent that increases SIRT4 level or activity. Such methods are useful, for example, for a subject that is malnourished or underweight.

In certain embodiments, the present invention relates to methods of reducing a subject's cholesterol level by administering an agent that inhibits SIRT4 level or activity. Such a method can be used to reduce the cholesterol level in a subject that has an above-normal cholesterol level. In some embodiments the subject has a total cholesterol level of above 180 mg/dL, above 200 mg/dL or above 240 mg/dL.

Also provided herein are methods of increasing SIRT1 activity in a cell by contacting the cell with a SIRT4 inhibitor. Increased SIRT1 activity has been demonstrated to prevent and treat many age related diseases. Such methods are therefore useful, for example, for treating SIRT1 related diseases, including, but not limited to, as age-related diseases, such as Type II Diabetes, cardiovascular disease and cancer.

### Pharmaceutical Compositions

Pharmaceutical compositions of this invention include any modulator identified according to the present invention, or a pharmaceutically acceptable salt thereof, and a pharmaceutically acceptable carrier, adjuvant, or vehicle.

Methods of making and using such pharmaceutical compositions are also included in the invention. The pharmaceutical compositions of the invention can be administered orally, parenterally, by inhalation spray, topically, rectally, nasally, buccally, vaginally, or via an implanted reservoir. The term parenteral as used herein includes subcutaneous, intracutaneous, intravenous, intramuscular, intra articular, intrasynovial, intrasternal, intrathecal, intralesional, and intracranial injection or infusion techniques.

Dosage levels of between about 0.01 and about 100 mg/kg body weight per day, preferably between about 0.5 and about 75 mg/kg body weight per day of the modulators described herein are useful for the prevention and treatment of disease and conditions. The amount of active ingredient that may be combined with the carrier materials to produce a single dosage form will vary depending upon the host treated and the particular mode of administration. A typical preparation will contain from about 5% to about 95% active compound (w/w). Alternatively, such preparations contain from about 20% to about 80% active compound.

### Kits

The present invention provides kits, for example for screening, diagnosis, preventing or treating diseases, e.g., those described herein. For example, a kit may comprise one or more polypeptides or one or more modulators, optionally formulated as pharmaceutical compositions as described above and optionally instructions for their use. In still other embodiments, the invention provides kits comprising one or more one or more polypeptides or one or more modulators, optionally formulated as pharmaceutical compositions, and one or more devices for accomplishing administration of such compositions.

Kit components may be packaged for either manual or partially or wholly automated practice of the foregoing methods. In other embodiments involving kits, this invention contemplates a kit including compositions of the present invention, and optionally instructions for their use. Such kits may have a variety of uses, including, for example, imaging, diagnosis, therapy, and other applications.

All publications, including patents, applications, and GenBank Accession numbers mentioned herein are hereby incorporated by reference in their entirety as if each individual publication or patent was specifically and individually indicated to be incorporated by reference. In case of conflict, the present application, including any definitions herein, will control.

The invention now being generally described, it will be more readily understood by reference to the following examples, which are included merely for purposes of illustration of certain aspects and embodiments of the present invention, and are not intended to limit the invention.

### EXEMPLIFICATION

### Experimental Procedures

### Mice and Diets

Unless otherwise specified, Male 129/Sv SIRT4 KO (Haigis et al., (2006) Cell 126, 941-954.) and WT litter-mates were used in the studies described herein. Mice were housed in 12 hour light-dark cycle (7 PM lights off and 7 AM lights on) in temperature controlled rooms. Unless specified as otherwise, Mice were maintained on a normal chow diet (Picolab diet 5053, energy content: 13% fat, 25% protein, 62% carbohydrates, Labdiet). High fat diet (D12492: 60% fat, 20% protein, 20% carbohydrates, Research diets) was provided for 16 weeks (n=6 per genotype) and a separate group of WT mice (n=6) were fed a low fat diet (D12450B: 10% fat, 20% protein, 70% carbohydrate, Research Diets). Food intake and body weight were measured weekly at 9 AM and daily food intake was analyzed during a period of 5 days each day at 9 AM. Fasting experiments, to measure sirtuin levels, were performed in 26-week-old male 129/Sv mice. Mice were fasted for 0, 10, 12 and 24 hours, starting the fast at 9 AM. Animal care and experiments were carried out in accordance with both institutional and federal animal care regulations and were approved by the Harvard Medical Area Standing Committee on Animals.

### Primary Cell Culture

Primary hepatocytes were isolated using a two-step perfusion protocol, based on previous methods (Lin et al., (2004) Cell 119, 121-135.). Briefly, livers were perfused first with Hanks balanced salt solution (HBSS, pH 7.4), containing glucose (1.0 g/l), EDTA (0.2 g/l), HCO₃ (2.1 g/l) and KCl (0.4 g/l) for 5 minutes. Next, livers were perfused for 15 min with a collagenase buffer (pH 7.4, Invitrogen). After perfusion, livers were dissected, minced, filtered, and hepatocytes purified using Percoll (Sigma) and plated (500,000 cells per well) on collagen coated 6 well plates (BD Biosciences) in DMEM (4.5 g/l glucose) containing 10% FBS, 2 mM pyruvate, 2% Pen/strep, 1 mM dexamethasone and 100 nM insulin. Typical primary hepatocyte isolations yielded 12-14×10⁶ cells per liver with 96%-98% cell viability (assessed by Trypan blue exclusion assay). Two hours after plating, medium was replaced with maintenance medium (DMEM with 0.2% BSA, 2 mM pyruvate, 2% Pen/strep, 0.1 mM dexamethasone and 1 nM insulin). Fatty acid oxidation assays were performed one day after isolation.

Mouse embryonic fibroblasts (MEFs) were isolated on embryonic day 12.5-14.5 from heterozygous females that were mated with heterozygous males. Primary MEFs were cultured in DMEM containing 10% FBS and 0.1 mM BME and were used between passages 2 and 5.

### Luciferase Assay

Transactivation assays were carried out in mouse H2.35 hepatoma cells and human HEK293T cells transfected with PPARα and RXRα. Cells were cultured in 60 mm dishes and transfected with pCMV, SIRT4, and SIRT4 mutant (H161A), using Lipofectamine 2000 reagent according to the protocol of the manufacturer. After 24 hours, cells were co-transfected with (PPRE)3-luciferase reporter vector (2 μg), or PGL3 FF-Luc reporter control, and Ren-Luc (200 ng). The following day, cells were transferred to 96 well plates. Following 12 hr treatment with WY14643 (0.5, 1 μM) or DMSO cells were lysed and assayed for luciferase activity (Luciferase assay system, Promega) and normalized to co-expressed renilla luciferase activity.

### Gene Expression

RNA was isolated from frozen liver tissue of overnight fasted mice or from cells using Trizol (Invitrogen) according to the manufacturer's instructions and further purified using RNeasy columns (Qiagen). Amplification and detection of target and reference cDNA samples was performed on a Lightcycler 480 (Roche) using Lightcycler 480 Sybr Green I Mastermix (Roche). A standard curve was generated for all genes using serial dilutions of a pool prepared from all cDNA samples. mRNA levels of target genes were normalized using beta 2 microglobulin (B2m), peptidyl-prolyl isomerase (Ppia) and ribosomal protein 16 (Rps16) as reference genes. Primer sequences are listed in Supplemental table S2.

### Microarray Analysis

SIRT4 KO and WT litter-mates (males, n=6 per genotype, 7-8 mo old littermates) were fasted overnight for 16 hours and were sacrificed by cervical dislocation. All samples were individually hybridized on Affymetrix Mouse Genome 430 2.0 GeneChips by the Biopolymers Facility (Harvard Medical School). Data analysis was performed using dCHIP software. Differentially expressed genes between WT and SIRT4 KO mice were ranked according to the dCHIP calculated p-value that takes into account measurement errors. ErmineJ (Lee et al., (2005) BMC Bioinformatics 6, 269.) was used to calculate overrepresentation of gene ontology terms in the data set using dCHIP p-values as gene scores. Transcriptome similarity analysis was performed based on previously published methods (Schumacher et al., (2008) PLoS Genet 4, e1000161). In brief, all analyzed datasets were downloaded from GEO or ArrayExpress and significantly differential expressed genes were compared to SIRT4 KO differentially expressed genes (p<0.1). To exclude confounding factors associated with between-platform and between-tissue comparisons, we selected adult mouse liver data sets that used Affymetrix GeneChip Mouse genome 430 2.0 or Affymetrix GeneChip Murine genome U74 platforms from Gene Expression Omnibus (GEO) and ArrayExpress. Similarity was scored using two criteria: a gene should be significantly different in both gene sets and the direction of regulation should be the same. Statistical significance was calculated by permutation among annotated unique genes using 10,000 permutations. All comparisons were performed in Microsoft Excel using Visual Basic to automate calculations.

### Western Blotting

Western blotting was performed using antibodies directed against phospho-ACC (Ser-79), ACC, phospho-AMPKα (Thr-172), AMPKα (Cell Signaling). Actin, Flag and HA antibodies were from Sigma. Antibodies raised against SIRT4 were described previously (Haigis et al., (2006) Cell 126, 941-954.).

### Fatty Acid Oxidation

Cells were incubated overnight in culture medium containing 100 μM palmitate (C16:0) and 1 mM carnitine. In the final 2 hours of incubation, cells were pulsed with 1.7 μCi [9,10(n)-³H]palmitic acid (GE Healthcare), and the medium was collected to analyze the released ³H₂O, formed during cellular oxidation of [³H]palmitate. In brief, medium was TCA precipitated, supernatants were neutralized with NaOH and loaded onto ion exchange columns packed with DOWEX 1×2-400 resin (Sigma). The radioactive product was eluted with water and quantitated by liquid scintillation counting. Oxidation of [³H]palmitate was normalized to protein content using Bio-Rad DC protein assay. Etomoxir, a specific inhibitor of CPT1a, was used to specifically inhibit mitochondrial fatty acid oxidation.

### Plasma and Liver Metabolic Parameters

Blood was collected from the tail vein of mice in EDTA coated microvette CB300 tubes (Sarstedt) and plasma was separated by centrifugation. Blood glucose was read directly from the tail vein using a glucose meter (OneTouch Ultra 2, Lifescan). Insulin was analyzed using Ultra Sensitive Mouse Insulin ELISA (Alpco). Non-esterified fatty acids (NEFA) in culture medium were analyzed using a commercial kit (WAKO diagnostics). GTT was performed after an overnight fast by injecting mice i.p. with 2 g/kg BW glucose and blood glucose was read from the tail vain using a glucose meter. Plasma NEFA, culture medium NEFA, triglycerides and total ketone bodies were analyzed using commercial kits (WAKO diagnostics). Liver triglycerides and liver fatty acids were analyzed by the Vanderbilt Mouse Metabolic Phenotyping Center (MMPC) Lipid Lab.

### NAD/NADH and ATP/ADP Analysis

NAD, ATP and ADP levels were analyzed in acid-soluble fractions from livers of SIRT4 WT and KO mice. For NAD analysis, frozen pulverized tissue was extracted with 7% cold perchloric acid and O¹⁸-NAD was used as internal control. Samples were neutralized with 3 M NaOH and 1 M phosphate buffer (pH˜9) and centrifuged before separation of NAD from other cellular components by HPLC. NAD peaks were collected according to the standard's retention time and dried on lyophilyzer. MALDI-TOF was used to detect distinct peaks (m/z=664 or 666) corresponding to isotopomers of NAD. Corrections were applied for isotopic abundance.

NADH levels were analyzed by extracting frozen pulverized livers in 0.05 M NaOH/1 mM EDTA by vortexing and sonication. Additionally, samples were incubated at 60° C. for 30 mins. After cooling on ice for 5 mins, and centrifuging, samples were neutralized with 0.1 M, 1 M HCl and 300 mM phosphate buffer (pH˜4.4). The neutralized sample was centrifuged and supernatant was used for enzymatic cycling assay measurements. The sample was mixed with cycling assay buffer containing 25 mM Tris-HCl (pH˜8), 5 mM MgCl₂, 50 mM KCl, 2.25 mM lactate, 54 μM resazurin and 0.4 u/mL lactate dehydrogenase. The cycling reaction was initiated with the addition of diaphorase and the increase in the resorufin fluorescence (with excitation at 560 nm and emission at 590 nm) was measured continuously on a fluorescent plate reader. The concentration of NADH was measured fluorometrically using the cycling assay described above. Standard curves were obtained by processing the standard NADH samples along with the biological samples.

ATP and ADP were analyzed according to previously published methods (Vander Heiden et al., (1999) Mol Cell 3, 159-167). In brief, acid-soluble fractions were neutralized with 2 M K₂CO₃ in 6 M KOH and centrifuged to precipitate insoluble perchlorate. The supernatant was used for ATP/ADP measurements using a luciferase-based assay (Biovision). Concentrations of ATP and ADP in samples were determined by using standard curves for ATP and ADP.

### Statistical Analysis

Analysis was performed using an unpaired Student's t test, and significant differences are indicated by a single asterisk when p<0.05 and double asterisks when p<0.01.

### Example 1

### SIRT4 is down-regulated during fasting

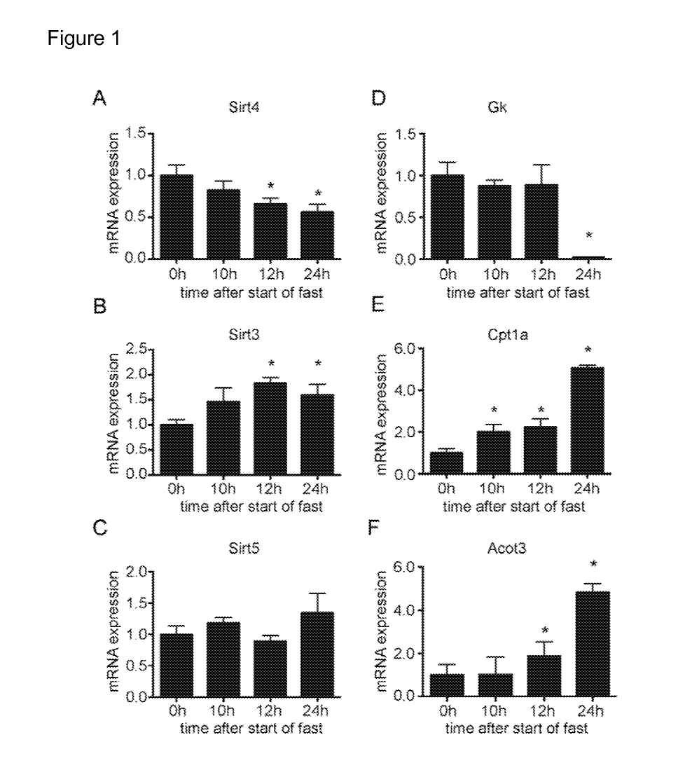

To investigate whether modulation of SIRT4 activity plays a role in the response to nutrient deprivation in the liver, SIRT4 gene expression levels in livers of fasted 129/Sv mice were analyzed by quantitative RT-PCR, as described above. The fasting period was initiated at the beginning of the light cycle (9 AM) and food deprivation was continued for 24 hours. At the onset of the dark cycle (the period when mice normally start eating), SIRT4 levels were down-regulated by 20% (t=10 h), and after a 24-hour fast the levels of SIRT4 transcripts were decreased by half compared with starting fed levels of SIRT4 (p<0.05) (FIG. 1A). Because SIRT3 and SIRT5 are also mitochondrial NAD-dependent sirtuins that could be involved in redundant regulation of liver metabolism, expression of SIRT3 and SIRT5 was also examined by quantitative RT-PCR. In contrast to the down-regulation of SIRT4 upon fasting, nutrient deprivation induced SIRT3 by 1.8-fold (p<0.05) (FIG. 1B), but did not significantly modulate SIRT5 levels (FIG. 1C). The 24-hour fasting period suppressed glucokinase (Gk) expression and increased expression of carnitine palmitoyltransferase 1a (Cpt1a) (FIG. 1D) and acyl-CoA thioesterase 3 (Acot3) 5.1-fold and 4.8-fold, respectively (FIGS. 1E and 1F), demonstrating that fasting induces a robust shift from glycolysis to fatty acid oxidation. Taken together, these results demonstrate that SIRT4 levels are down-regulated in wild-type livers during fasting and implicate that SIRT4 participates in the shift in liver metabolism during nutrient deprivation.

### Example 2

### Loss of SIRT4 Enhances Lipid Catabolism Gene Expression Upon Nutrient Deprivation

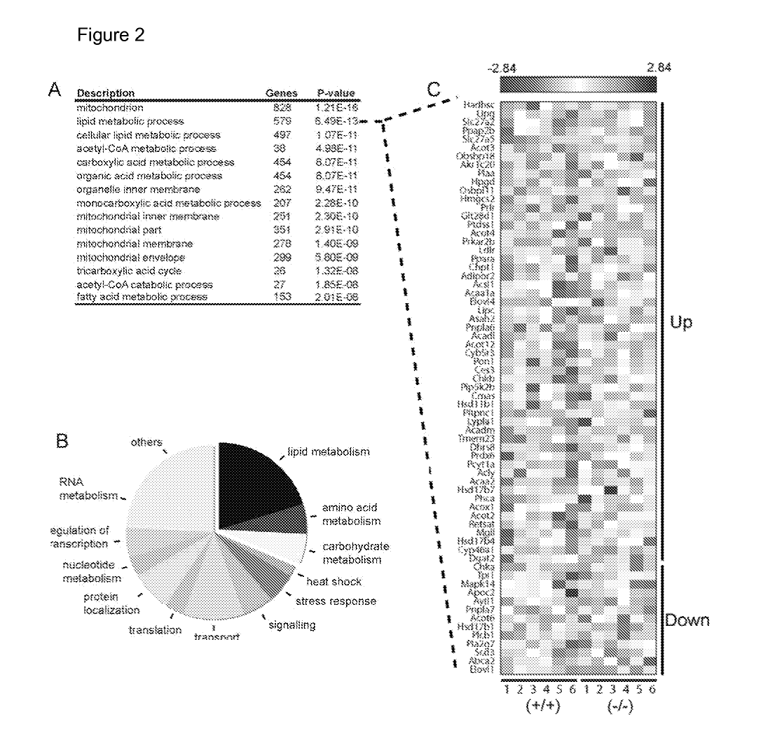

To characterize physiological pathways regulated by SIRT4 in the liver upon fasting, genome-wide gene expression profiles in SIRT4 knockout (KO) and SIRT4 wild-type (WT) mouse livers from 16-hour-fasted mice were analyzed by microarray analysis, as described above. SIRT4 KO mice are developmentally normal with no obvious liver phenotype (Haigis et al., (2006) Cell 126, 941-954). Analysis of the data revealed that hepatic gene expression profiles of SIRT4 KO mice (n=6) were only subtly different than those of SIRT4 WT mice (n=6). Out of the 22,094 unique genes on the microarray, only 654 genes were significantly different between SIRT4 KO and WT mice (p<0.05). Nevertheless, detailed analysis of over-represented pathways using ErmineJ clearly revealed that the majority of differentially expressed genes in the SIRT4 KO livers encoded mitochondrial proteins (FIG. 2A). Moreover, metabolic pathways, including lipid, acetyl-CoA and tricarboxylic acid metabolism, were highly enriched (FIG. 2A). These data reinforce further support that SIRT4 is involved in the regulation of liver metabolic programs, even at the transcriptional level. Although detailed functional classification of the significantly changed genes (p<0.01) demonstrated that 31% of the most significantly changed genes encoded proteins involved in metabolism of lipids, amino acids and carbohydrates (FIG. 2B), other cellular processes, like transport (11%) and RNA metabolism (9%) were also found to be overrepresented (FIG. 2B). Overall, lipid metabolism genes accounted for 20% of the most significantly changed genes, the majority of which were up-regulated by loss of SIRT4 (FIGS. 2B and 2C).

The above described gene expression analysis further revealed a coordinated up-regulation of fatty acid catabolic gene expression in SIRT4 KO mice. For example, expression of beta oxidation genes (Acadm, Acadl, Hadhcs, Acaa1a, Acaa2, Acox1), lipases (Lipg, Lipc) and thioesterases (Acot2, Acot3, Acot4) was all enhanced by loss of SIRT4 (FIG. 2C). Consistent with a profile of increased fatty acid catabolism, genes encoding proteins involved in fatty acid synthesis were suppressed (Elovl, Scd3, Abca2).

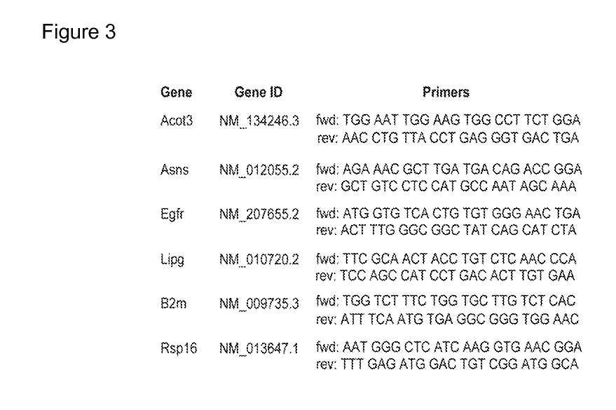

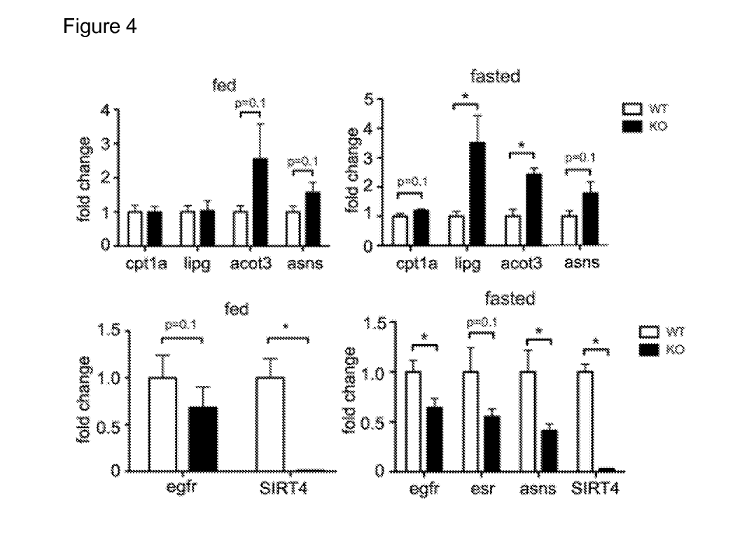

Because loss of SIRT4 intensified the lipid fasting response in livers, the expression of Cpt1a, Lipg and Acot3 were analyzed in fed and fasted SIRT4 KO mice by quantitative RT-PCR using the primers indicated in FIG. 3. It was found that Acot3 and Asns were elevated in SIRT4 KO animals under both fed and fasted conditions, indicating that there is inappropriate lipid catabolism in the fed state (FIG. 4). The levels of Lipg and Cpt1a were only elevated during fasting in SIRT4 KO liver (FIG. 4). The levels of Egfr and Esr were decreased under fed and fasted conditions, demonstrating a down-regulation of growth signaling pathways under conditions of high lipid catabolism (fasting). In sum, the microarray data analysis indicates a focused, coordinated metabolic shift in SIRT4 KO liver towards fatty acid catabolism.

### Example 3

### Loss of SIRT4 Up-Regulates PPARα-Dependent Transcription of Lipid Catabolism Genes

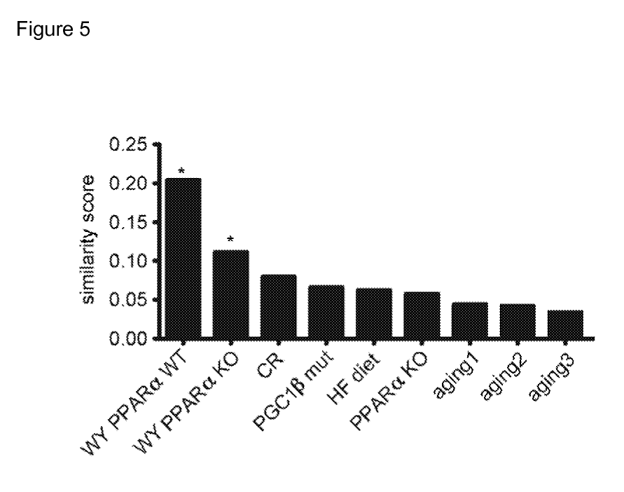

PPARα is the major transcriptional activator of fatty acid catabolism during fasting (Kersten et al., (1999) J Clin Invest 103, 1489-1498; Leone et al., (1999) Proc Natl Acad Sci USA 96, 7473-7478). Therefore, whether SIRT4 regulates PPARα-dependent transcriptional activity was examined by comparing the gene expression profile of SIRT4 KO livers with published liver gene expression profiles from PPARα KO mice and WT mice treated with WY14643, a chemical agonist of PPARα activity. A significant overlap between gene expression profiles of PPARα activation (mice treated for 5 days with WY14643, GSE8295) and SIRT4 KO expression profiles was observed. In contrast, the similarity was much lower in PPARα KO mice treated with WY14643 (FIG. 5) and the transcriptome of PPARα KO mice without activation by WY14643 did not overlap with the SIRT4 KO transcriptome (FIG. 5). Furthermore, the SIRT4 KO gene expression profile did not significantly overlap with differential liver gene expression profiles from PGC-1β mutant, caloric restriction (CR), high fat diet, and aging transcriptomes (FIG. 5), demonstrating a unique and specific overlap between gene expression changes in SIRT4 KO mice and in mice where PPARα is activated. These results suggest a coordination of SIRT4 down-regulation during fasting with concomitant PPARα activation, an idea we sought to investigate in greater depth.

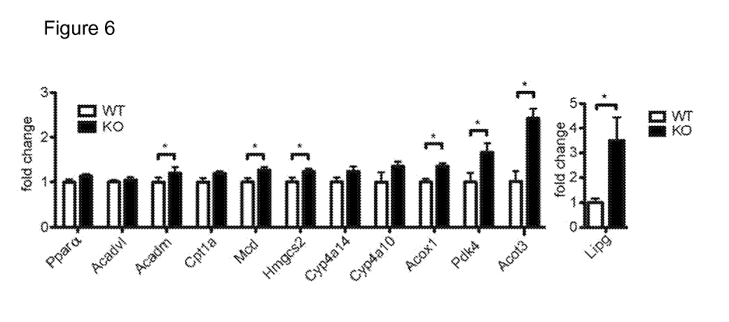

To test in a more directed manner whether PPARα activation in fasted SIRT4 KO mice is heightened, a set of canonical PPARα target genes were analyzed by quantitative real time RT-PCR (Mandard et al., (2004) Cell Mol Life Sci 61, 393-416). The PPARα target genes (Lipg, Acot3, Pdk4, Acox1, Hmgcs2, Mcd and Acadm) were significantly elevated by 1.3-3.5 fold in the livers of fasted SIRT4 KO mice as compared to the livers of fasted WT mice (FIG. 6). All other PPARα target genes analyzed (Acadvl, Cpt1a, Cyp4a14 and Cyp4a10) were also at least marginally induced. Interestingly, no induction of Pparα itself was detected (FIG. 6), thus ruling out regulation of PPARα targets through induction of Pparα gene expression. These findings are consistent with the microarray studies and demonstrate that a network of PPARα targets is up-regulated by loss of SIRT4.

Collectively, the differences in gene expression between wild-type and SIRT4 KO livers indicate a physiological up-regulation of breakdown of acyl-CoAs and triglycerides to FFA in SIRT4 deficient mice. The gene expression differences also indicate a subsequent increase in fatty acid oxidation and down-regulation of amino acid and protein synthesis. Carbons from fatty acids are further metabolized to acetyl-CoA and enter into the TCA cycle or are used for ketone production. These results are consistent with findings that GDH activity is elevated in SIRT4 KO mice because, like PPAR-α, GDH activity also increases during periods of fasting. Like PPAR-α, GDH activity increases in wild-type animals during CR.

### Example 4

### PPARα activation is suppressed by SIRT4

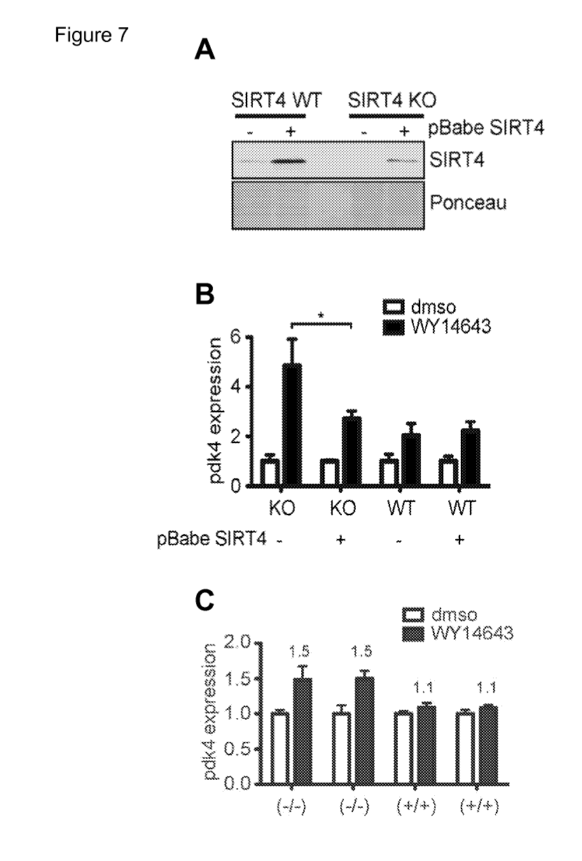

The results of the gene expression assays described above indicate that SIRT4 represses PPARα activity and fatty acid catabolism, and thus suggests down-regulation of SIRT4 in fasting promotes PPARα activity and fatty acid catabolism. To determine whether SIRT4 is a bona fide repressor of PPARα in a cell-autonomous manner, the induction of the PPARα target gene Pdk4 was examined in SIRT4^{-/-} and SIRT4′^{+/+} mouse embryonic fibroblast (MEF) cell lines exposed to the PPARα agonist WY14643 (WY), as described above. In wild-type cells, activation of PPARα by WY14643 leads to a 2-fold increase in Pdk4 expression (FIG. 7B). By contrast, the stimulation of Pdk4 expression was more than doubled in MEFs lacking SIRT4, compared with WT cells (FIGS. 7B and 7C). This suggests that SIRT4 negatively regulates the ability of PPARα to activate transcription of Pdk4. To test this hypothesis further, the wild-type and SIRT4^{-/-} MEFs were reconstituted with a retroviral expression vector for SIRT4 (FIG. 7A). Reconstitution of SIRT4 in previously null MEFs suppressed the induction of Pdk4 by WY14643, but had no effect on the induction of Pdk4 by WY14643 in wild-type MEFs (FIG. 7B). Thus, SIRT4 represses PPARα activation in a cell-autonomous manner.

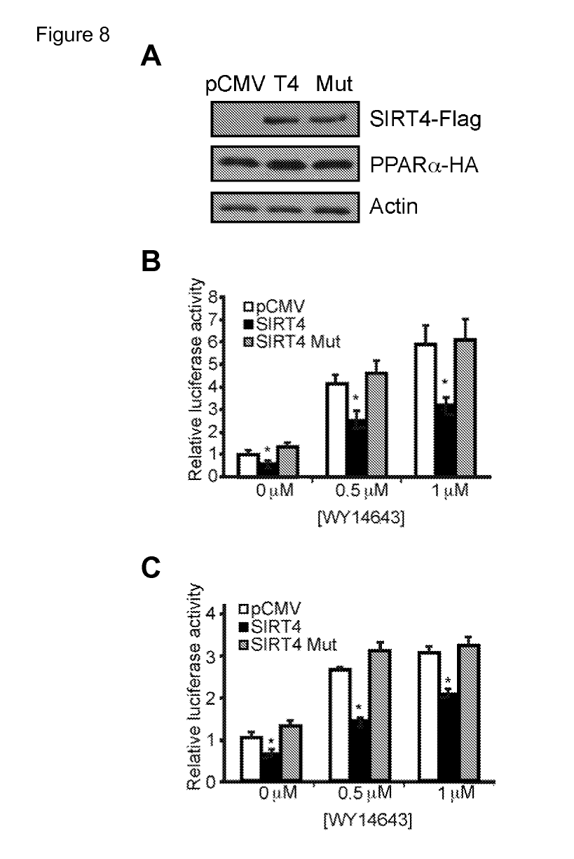

To further test the connection of PPARα transcriptional activity control by SIRT4, PPARα transcriptional activity was examined using luciferase reporter assays in cells with or without increased levels of SIRT4. Human embryonic kidney (HEK293T) cells expressing elevated levels of either SIRT4 or the enzymatically inactive SIRT4 (H161A) mutant protein, were transfected with a luciferase reporter driven by three tandem repeats of a consensus PPAR response element (3×PPRE), together with constructs expressing PPARα, RXRα and a control Renilla luciferase reporter (FIG. 8A). Increased levels of wild type SIRT4 significantly reduced WY induced transactivation of PPARα in a dose-dependent manner (FIG. 8B). By contrast, HEK293T cells that expressed H161A-SIRT4 had similar PPARα reporter activity compared to cells transfected with the pCMV control plasmid (FIG. 8B).

H2.35 mouse hepatoma cells were used to investigate the effect of SIRT4 on PPARα activity in hepatic cells that endogenously express PPARα and RXRα. Consistent with the reduction of PPARα activity by SIRT4 in human HEK293T cells (FIG. 8B), SIRT4 reduced 3×PPRE reporter activity in mouse hepatoma cells as well, whereas H161A-SIRT4 did not block PPARα promoter activity (FIG. 8C). Taken together, these results validate the model that the enzymatic activity of SIRT4 is required to achieve repression of PPARα activity in multiple cell types.

### Example 5

### Fatty Acid Oxidation is Increased in Primary Cells from SIRT4 KO Mice

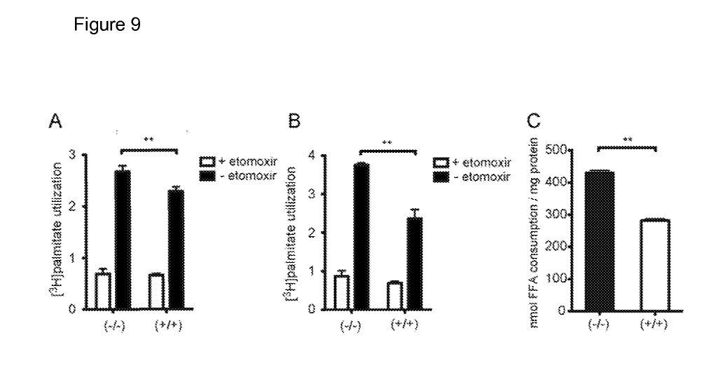

Ultimately, the up-regulation of lipid catabolism gene expression during nutrient deprivation leads to enhanced rates of fatty acid oxidation. Based on the observations that SIRT4 represses PPARα activity and expression of PPARα target genes, it is likely that decreased levels of SIRT4 induce increased fatty acid oxidation from cells. To test this model, the rates of oxidation of palmitate, a saturated long chain fatty acid, were analyzed in primary MEFs and primary hepatocytes isolated from SIRT4 WT and KO mice. The CPT1a inhibitor, etomoxir, (Baht and Saggerson, 1989) was used to specifically block mitochondrial import of fatty acids. The results of these assays indicated that oxidation rates were 17% higher (p<0.05) in SIRT4 KO MEFs than in WT MEFs (FIG. 9A), and that etomoxir inhibited fatty acid oxidation strongly in both cell types. Furthermore, in primary hepatocytes isolated from SIRT4 KO mice higher rates (59%, p<0.01) of oxidation of palmitate than in hepatocytes from WT mice were observed (FIG. 9B). Etomoxir effectively blocked mitochondrial fatty acid oxidation in both WT and SIRT4 KO hepatocytes (FIG. 9B).

The effect of SIRT4 on fatty acid oxidation was also determined by analyzing the levels of fatty acids in culture medium before and after exposure to palmitate. Consumption of palmitate from the culture medium was significantly higher in SIRT4 KO hepatocytes than in WT hepatocytes, confirming that utilization of palmitate was indeed higher in SIRT4 KO hepatocytes (FIG. 9C). Thus, decreased SIRT4 in cells ex vivo enhances fatty acid uptake and increases rates of fatty acid oxidation. These data functionally demonstrate that SIRT4 acts as an upstream regulator of fatty acid utilization and oxidation pathways and confirm that SIRT4 represses PPARα, a positive regulator of fatty acid oxidation pathways.

### Example 6

### SIRT4 KO mice on a low fat diet have normal lipid homeostasis and body weights

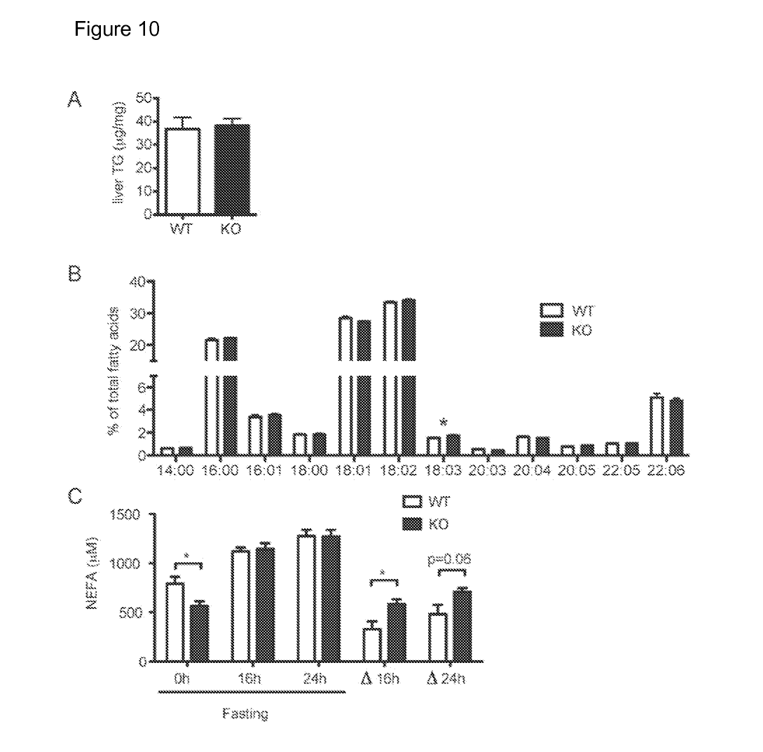

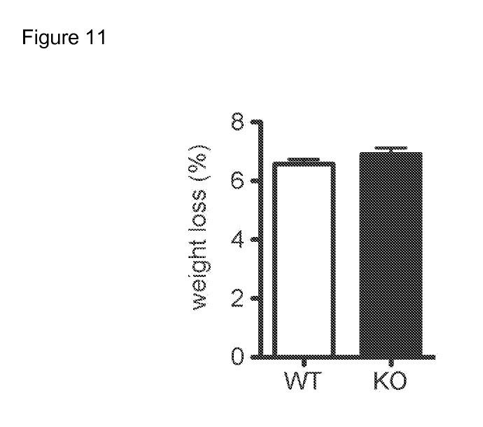

Because PPARα is activated by fatty acids, the possibility that altered lipid metabolism profiles could be responsible for the induction of PPARα activity in livers of SIRT4 KO mice was investigated. However, total liver triglyceride levels were not different between SIRT4 KO and WT mice (FIG. 10A) and fatty acid profiles of the triglyceride fraction in the liver were also comparable (FIG. 10B), suggesting lipid profiles are not likely to control differences observed in PPARα activity. Consistently, fasting plasma NEFA levels were similar between SIRT KO and WT mice after a 16 h and 24 h fast (FIG. 10C). On the other hand, pre-fasted fatty acid levels in SIRT4 KO mice were lower (562±μM) than those in SIRT4 WT mice (792±70 μM), establishing that, overall, the SIRT4 KO mice circulate more fatty acids during fasting than do WT mice (FIG. 10C). Weight loss during fasting of SIRT4 KO and WT mice on a standard low fat diet was not different (FIG. 11).

### Example 7

### SIRT4 Loss Protects Against Dietary Induced Weight Gain

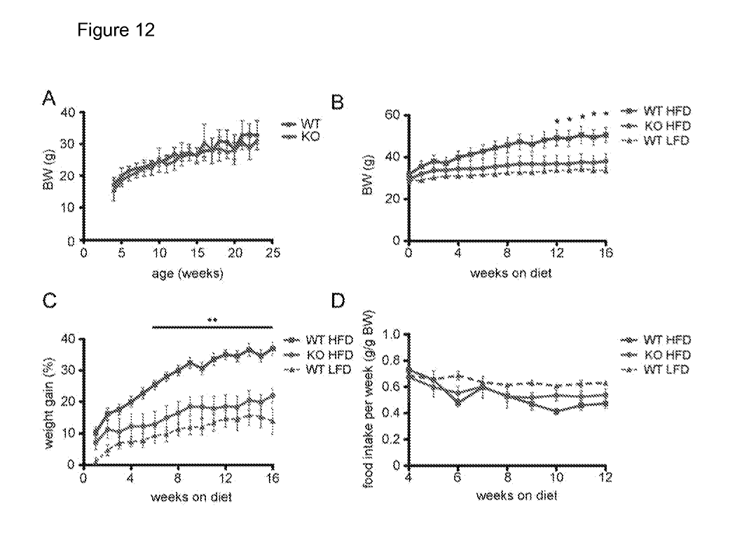

Despite the fact that elevated fatty acid catabolism in SIRT4 KO mice could affect the body weight of SIRT4 KO mice, no differences in body weights of mice fed a standard low fat diet (LFD) up to 6 months of age were observed (FIG. 12A). However, the repression of PPARα activity by SIRT4 described above suggests that SIRT4 may function to regulate fat metabolism during dietary stress. Furthermore, SIRT4 KO mice have higher expression of fatty acid oxidation genes, especially in the fasting state.

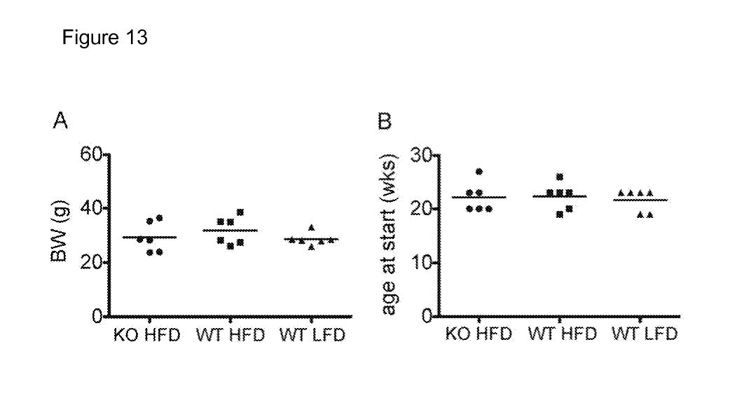

Ironically, in order to increase the turnover of the high burden of fatty acids, the livers of mice fed a high fat diet (HFD) up-regulates a similar transcriptional programs to when they are in a fasting state (Savage et al., (2007) Physiol Rev 87, 507-520). In order to determine how SIRT4 suppression effects mice under these conditions, SIRT4 KO mice and WT controls (males, n=6 per group, FIGS. 13A and 13B) were fed a HFD, consisting of 60% of calories originating from fat. A HFD was maintained for 16 weeks and a control group of WT mice was maintained on a LFD (10% of total calories from fat) during the 16 week period. After 16 weeks on the HFD the body weight of the WT mice increased to 50.1±3.6 g (mean±SEM) (FIG. 12B), representing a 37% increase in starting body weight (FIG. 12C). This value was statistically higher than WT control mice fed a LFD (14% increase in starting body weight) (FIG. 12C). In contrast, the body weight of SIRT4 KO mice on a HFD after 16 weeks was 38.0±3.4 g (mean±SEM) (FIG. 12B), representing only a 22% increase in starting body weight, which was similar to WT control mice fed a LFD (FIGS. 12B and 12C). Taken together, these results show that loss of SIRT4 activity protects against weight gain when on a HFD.

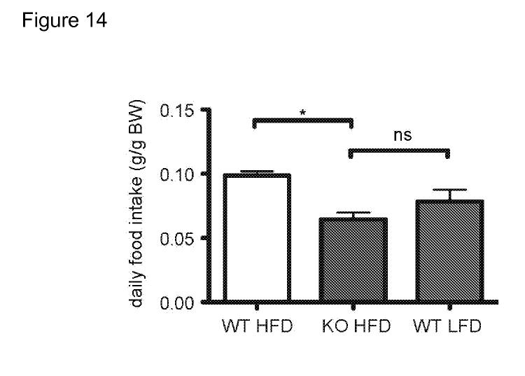

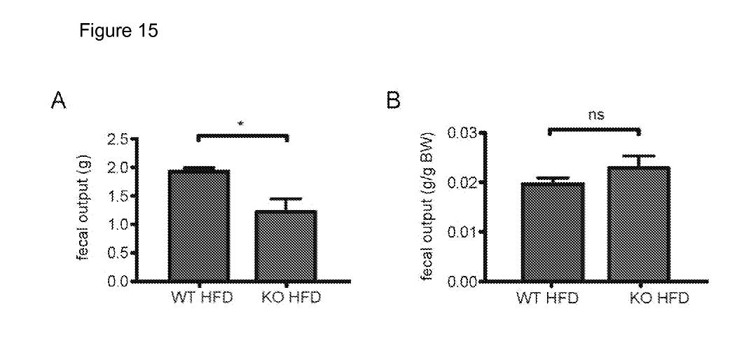

Decreased weight gain is often associated with decreased food intake. Therefore, weekly food consumption in animals from all three diet groups was also analyzed. SIRT4 KO mice on a HFD did not eat less food than the WT HFD mice. If anything, their food intake was slightly higher (FIG. 12D). Additionally, when we analyzed daily food intake over a period of 5 days, SIRT4 KO mice consumed similar amounts of food to the WT mice (FIG. 14). The mice fed a LFD consumed more food than mice on a HFD (FIG. 12C and FIG. 14), which is in accordance with the lower energy density of the LFD (3.85 kcal/g food) than the HFD (5.24 kcal/g food). In addition, total fecal output and relative fecal output in the SIRT4 KO and WT mice were not different (FIGS. 15A and 15B). Thus, overall lower body weight gain of SIRT4 KO mice cannot be attributed to differences in food intake or gastro-intestinal food uptake between SIRT4 KO and WT mice.

### Example 8

### SIRT4 KO Mice on a HFD Retain a Lean Physiology

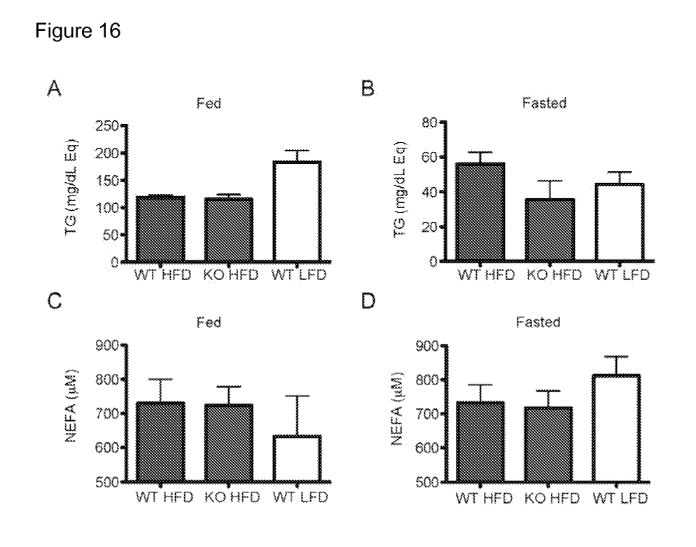

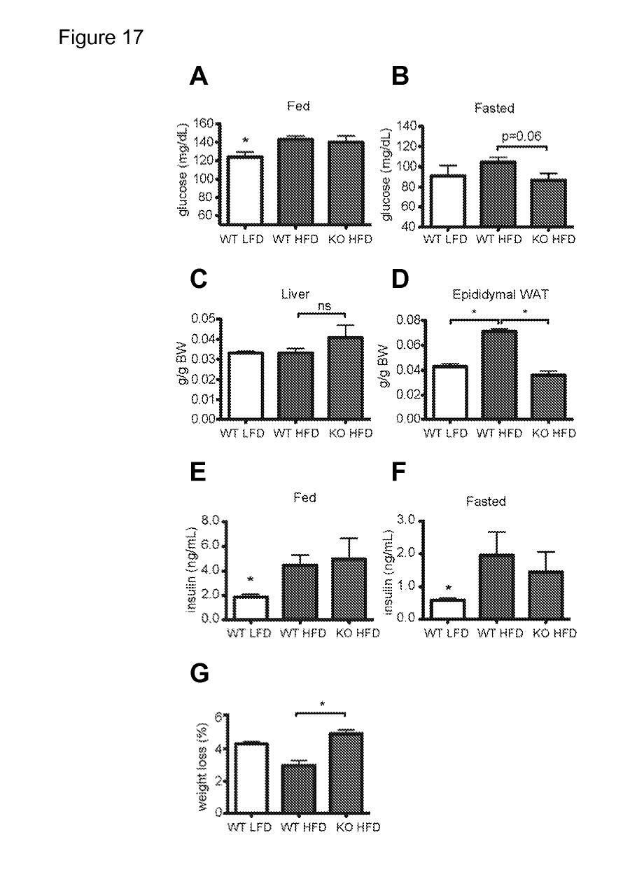

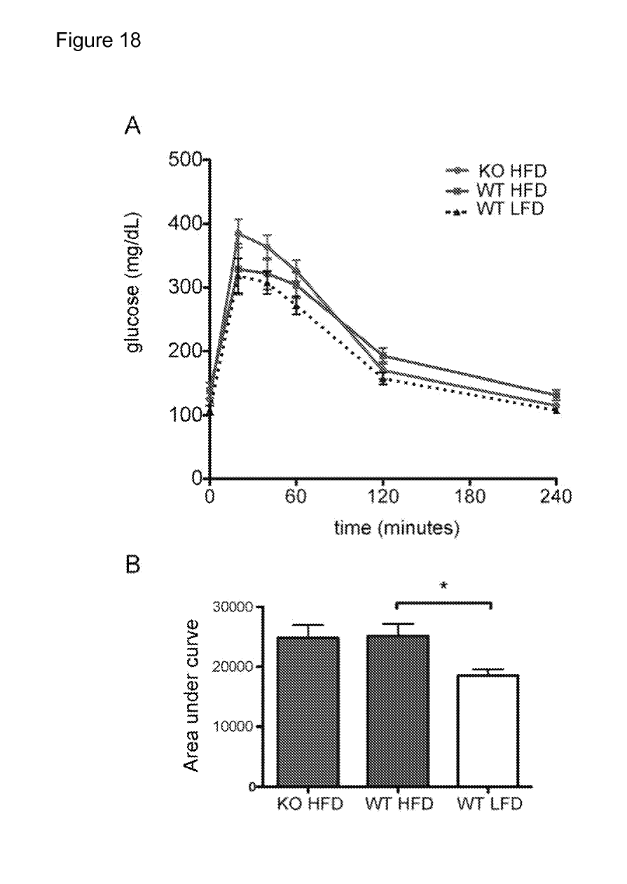

HFD is associated with increases in plasma lipid levels and hyperglycemia (Almind and Kahn, 2004). Moreover, depending on the strain of mice and the severity and duration of the HFD, mice may develop a diabetic state of insulin resistance and hyperglycemia (Almind and Kahn, 2004). To investigate whether loss of SIRT4 improves lipid and glucose homeostasis, plasma lipid and glucose parameters in SIRT4 KO and WT mice on a HFD were analyzed. Plasma triglyceride (FIG. 16A), NEFA (FIG. 16C), and ketone body levels were not significantly different between SIRT4 KO and WT mice in a fully fed state. In addition, blood glucose (FIG. 17A), plasma insulin (FIGS. 17E and 17F) and glucose clearance (FIG. 18) were comparable between WT and KO mice fed a HFD. On the other hand, after an overnight fast, SIRT4 KO mice on a HFD had lower glucose levels (86.7±6.9 mg/dL) than did WT mice on a HFD (104.3±4.8 mg/dL) (FIG. 17B).

Because SIRT4 KO mice on a HFD have lower body weights than WT mice on a HFD and demonstrate improved fasting lipid and glucose homeostasis, the weights of the liver and adipose tissue after the 16 week HFD period were examined. Liver weights were not significantly different between KO and WT mice on a HFD (FIG. 17C), but epididymal white adipose tissue weight was significantly lower in the SIRT4 KO mice (FIG. 17D). The latter was comparable to epididymal fat pad weight of WT mice on a LFD (FIG. 17D). Interestingly, when SIRT4 KO and WT animals were fasted, the overnight weight loss was significantly greater in SIRT4 KO mice (4.8%±0.7%) than in WT mice (3.0%±0.4%) (FIG. 17G). This indicates that SIRT4 KO mice do not conserve their fat stores and thus lose more weight when fasted overnight. In summary, long term HFD feeding or fasting in SIRT4 KO mice may trigger a state of induced fatty acid oxidation, which is characterized by protection from dietary induced weight gain with loss of SIRT4.

### Example 9

### Mechanism of action of SIRT4

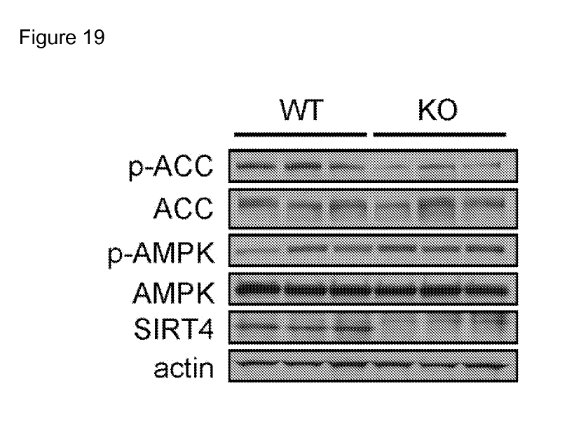

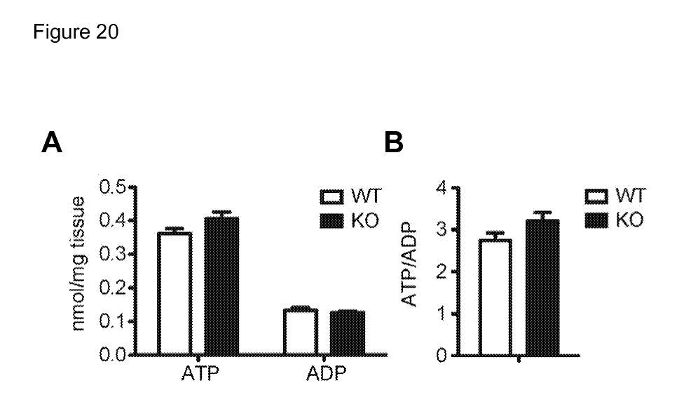

The mechanism through which SIRT4 mediates repression of PPARα and suppresses fatty acid oxidation was examined. Since PPARα is stimulated by phosphorylation under conditions of high AMP-activated protein kinase (AMPK) activity (Lee et al., (2006) Biochem Biophys Res Commun 340, 291-295), it was tested whether the increase of fatty acid oxidation that resulted from the loss of SIRT4 is caused by activation of AMPK. AMPK is activated by low ATP/ADP ratio and triggers fatty acid catabolism by phoshorylating and inhibiting acetyl-CoA carboxylase (ACC) (Kahn et al., (2005) Cell Metab 1, 15-25). The results of this assay indicated that levels of phosphorylated ACC were lower in fasted livers of SIRT4 KO mice than in SIRT4 WT mice (FIG. 19), suggesting down-regulation of AMPK. ATP and ADP levels were then analyzed in fasted SIRT4 KO mouse livers. Although ATP and ADP levels per se were not significantly different between SIRT4 KO and WT livers (FIG. 20A), the ATP/ADP ratio was slightly higher in SIRT4 KO mice (3.2±0.2) as compared to WT mice (2.7±0.18) (FIG. 20B), consistent with the decreased phosphorylation of ACC. These data indicate that AMPK signaling is not responsible for the enhanced fatty acid oxidation phenotype in SIRT4 KO mice.

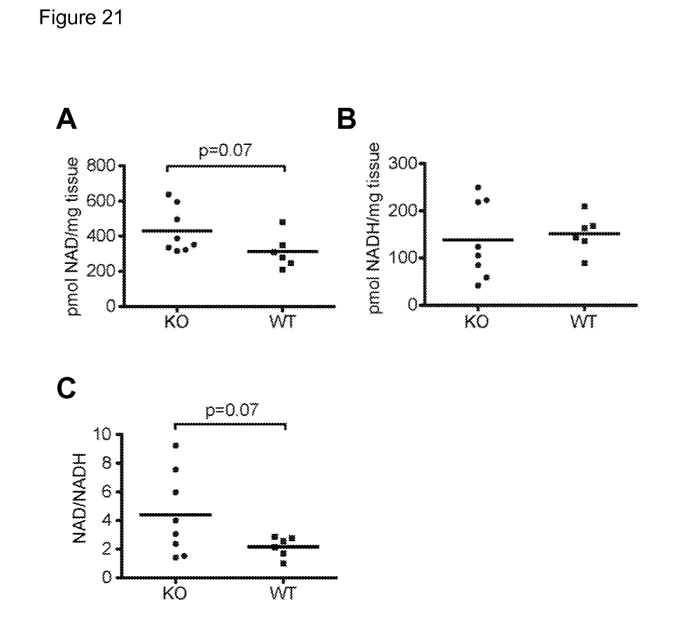

Next, it was examined whether SIRT4 could alter nuclear transcription of fatty acid oxidation enzymes by altering cross talk between the mitochondria and the nucleus. It was tested whether SIRT4-regulated metabolic intermediates from the mitochondria could impact PPARα dependent gene transcription. Enzymatic activity of SIRT4 depends on NAD, and other sirtuins have been shown to be regulated by the levels of NAD and NADH (Guarente and Picard, 2005). Interestingly, NAD levels were higher in SIRT4 KO livers after fasting (KO: 430±129 pmol/g tissue and WT: 312±95 μmol/g tissue) (FIG. 21A), while NADH levels were not significantly different (FIG. 21B). This resulted in higher NAD/NADH ratios in livers of SIRT4 KO mice (4.4±2.9) as compared to WT mice (2.2±0.7) (FIG. 21C). This suggests that the metabolite NAD could be one of the signals arising from the mitochondria to trigger enhanced nuclear transcriptional activity in response to SIRT4 suppression. As NAD concentration impacts sirtuin activity directly, loss of SIRT4 could activate other sirtuins or NAD-dependent pathways by increasing intracellular levels of NAD.

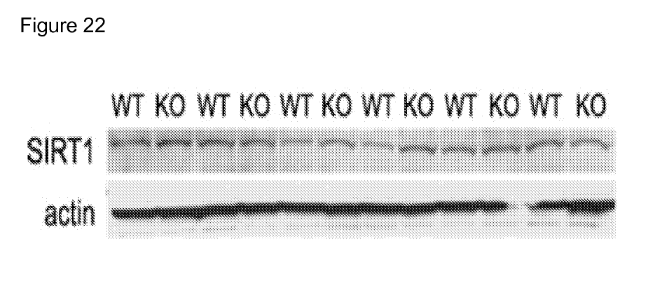

Notably, sirtuin SIRT1 has been shown to regulate co-activators and co-repressors of PPAR transcription factors. Though the protein level of SIRT1 is normal in SIRT4 KO mice (FIG. 22), it is likely that increased NAD observed in SIRT4 KO mice (FIG. 21A) promotes SIRT1 deacetylase activity. For example, it is known that PGC-1α is deacetylated by SIRT1 during nutrient deprivation, enhancing gluconeogenesis (Rodgers et al., (2005) Nature 434, 113-118). In addition, SIRT1 induces lipolysis in adipose cells by docking with co-repressors of PPARγ, a master regulator of adipose cell development (Picard et al., (2004) Nature 429, 771-776). Since PGC-1α is deacetylated by SIRT1 during nutrient deprivation (Rodgers et al., (2005) Nature 434, 113-118), it is probable that increased NAD observed in SIRT4 KO mice promotes SIRT1-mediated deacetylation of PGC-1a, thereby activating PPARα. Consistent with this model, SIRT1 increases mitochondrial fatty acid oxidation in liver and muscle cells, via PGC-1α deacetylation (Rodgers et al., (2005) Nature 434, 113-118).

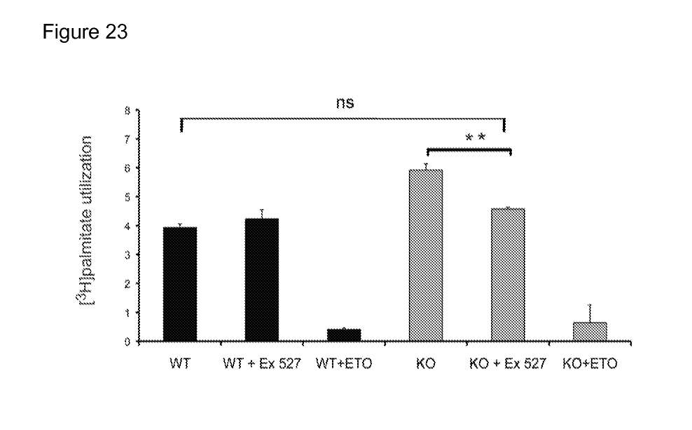

To confirm that SIRT4 suppresses Fatty Acid Oxidation through the suppression of SIRT1 activity, primary hepatocytes were isolated from WT or SIRT4 null mice and assayed for Fatty Acid Oxidation in the presence or absence of the SIRT1 inhibitor Ex 527. Confirming the results presented in FIG. 9B, in the absence of Ex 527 the SIRT1 KO hepatocytes exhibited significantly higher levels of Fatty Acid Oxidation than the WI hepatocytes. Addition of the SIRT1 inhibitor to the WT hepatocytes did not significantly alter the rate of Fatty Acid Oxidation (FIG. 23). This result is likely due to the fact that, in these cells, SIRT1 activity is already inhibited by SIRT4, and therefore addition of a SIRT1 inhibitor has no effect. On the other hand, when Ex 527 is added to SIRT4 KO hepatocytes the level of Fatty Acid Oxidation is significantly reduced (FIG. 23). That a SIRT1 inhibitor is able to inhibit Fatty Acid Oxidation in SIRT4 KO hepatocytes, but not in WT hepatocytes, indicates that SIRT1 is active and contributing to Fatty Acid Oxidation in the SIRT4 KO hepatocytes, but is suppressed in the SIRT4 WT hepatocytes. Thus, the results presented herein indicate that suppression of SIRT4 results in activation of SIRT1, likely through the elevation of cellular NAD levels.

### Example 10

### SIRT4 Directly Regulates Fatty Acid Oxidation and ATP Production by Hepatocytes

Mitochondria utilize both fatty acids and amino acids to contribute to electron transport and ATP production, but many questions remain about the regulation of this process and the cross-talk between fatty acid and amino acid metabolism. SIRT4 is a regulator of both nutrient pathways. SIRT4 is a mitochondrial ADP-ribosyltransferase that inhibits GDH activity (impacting amino acid metabolism) and suppresses the expression of genes that control fatty acid oxidation. SIRT4 therefore directly down-regulates fatty acid metabolism and controls ATP production from amino acids or fatty acids

SIRT4 KO mice display a coordinated up-regulation of genes involved in fatty acid breakdown (FIG. 2). SIRT4 KO MEFs demonstrate a stronger response to PPAR-a agonists than wild-type MEFs (FIG. 7). Data in cultured cells demonstrate that SIRT4 over-expression suppresses PPAR-a transcriptional activity (FIG. 8). SIRT4 thus suppresses fatty acid oxidation. The mechanism through which SIRT4 suppresses fatty acid oxidation is further tested by measuring fatty acid oxidation from isolated SIRT4 WT or KO hepatocytes, and using drugs and/or mediators of RNA interference (RNAi) to probe the mechanisms behind these changes. These studies involve isolating primary hepatocytes from wild-type or SIRT4 KO livers, measuring rates of palmitate oxidation, and performing assays in the presence of drugs and/or mediators of RNA interference that perturb fatty acid uptake, mitochondrial function or PPAR-a activity.

Primary hepatocytes are isolated using a two-step perfusion protocol that we have optimized, based on a previous method. Briefly, the livers are perfused first with Hanks balanced salt solution (HBSS, pH 7.4), containing glucose (1.0 g/l), EDTA (0.2 g/l), HCO₃ (2.1 g/l) and KCl (0.4 g/l) for 5 minutes. Next, livers are perfused for 15 min with a collagenase buffer (pH 7.4, Invitrogen). After perfusion, livers are dissected, minced, and hepatocytes purified using Percoll (Sigma) and plated (500,000 cells per well) on collagen coated 6 well plates in DMEM (4.5 g/l glucose) containing 10% FBS, 2 mM pyruvate, 2% Pen/strep, 1 mM dexamethasone and 100 nM insulin. Two hours after plating, medium are replaced with maintenance medium (DMEM with 0.2% BSA, 2 mM pyruvate, 2% Pen/strep, 0.1 mM dexamethasone and 1 nM insulin).

To examine the role of SIRT4 in fatty acid catabolism, primary hepatocytes are incubated with tritiated palmitate, a long chain fatty acid (C16:0), and its oxidation is measured by quantitating the radioactive product (³H₂O). Freshly isolated primary SIRT4 WT or KO hepatocytes are used after they have been incubated for one day in maintenance medium. Cells are then incubated overnight in maintenance medium containing 100 uM palmitate and 1 mM carnitine. In the final 2 hours of incubation, cells are pulsed with 1.7 uCi [9,10(n)-³H]palmitate (GE Healthcare), and the medium is collected to analyze the released ³H₂O formed during oxidation of [³H]palmitate. In brief, medium is TCA precipitated, and supernatants are neutralized with NaOH and loaded onto ion exchange columns packed with DOWEX 1×2-400 resin (Sigma). The radioactive product is eluted with water and quantitated using a scintillation counter (Beckman LS6500, available in the Pathology Department). Oxidation of [³H]-palmitate is normalized to protein content using Biorad DC protein assay, and data are represented as fatty acid oxidized/h/mg protein. Experiments are performed in at least triplicate, comparing results from at least 6 individual SIRT4 WT and KO hepatocyte isolations. When the rate of palmitate oxidation was measured from SIRT4 wild-type and KO hepatocytes and it was found that SIRT4 KO hepatocytes displayed a higher rate of fatty acid oxidation. Importantly, this result demonstrates that the changes in lipid catabolic gene expression have a biological function. This result also demonstrates that SIRT4 represses fatty acid oxidation and shows the utility of this system to explore mechanisms using drugs or RNAi. Also measured is the oxidation of short chain fatty acids, such as butyrate, and a medium chain fatty acid, such as octanoate.

Using isolated hepatocytes, the following types of drugs are used in the fatty acid oxidation studies in order to provide mechanistic insight and: 1) dissect the contribution of mitochondria versus peroxisomes in fatty acid catabolism, 2) investigate the role of PPAR-a and 3) investigate the role of sirtuins. First, drugs that inhibit mitochondrial fatty acid import or mitochondrial respiration are used. To inhibit fatty acid transport into the mitochondria, cells are pre-incubated with etomoxir, an inhibitor of the mitochondrial fatty acid transporter CPT1 or L-aminocarnitine, an inhibitor of CPT2. KCN, an inhibitor of mitochondrial electron transport, which has also been used to block fatty acid oxidation is also used. These drugs block mitochondrial fatty acid oxidation, leaving peroxisomal oxidation intact.

Also the results presented above demonstrate that SIRT4 represses gene expression through PPAR-α. According to this model, PPAR-α function contributes to the increased palmitate oxidation that was observed in hepatocytes from SIRT4 KO mice. Palmitate-driven beta oxidation studies are performed in hepatocytes that have been pre-incubated with either a PPAR-α activator (WY14643) or the PPAR-α inhibitor MK886. WY14643 studies are performed using DMSO treatment in parallel for a negative control.

The results presented herein indicate that sirtuins likely mediate the elevated fatty acid oxidation observed by loss of SIRT4. For these studies, hepatocytes are pre-incubated with drugs that block general (all sirtuins) and specific sirtuin enzymatic activity. Chemical compounds such as nicotinamide and sirtinol are used, which have been found to inhibit ADP-ribosyltransferase and deacetylase activities. Thus, these compounds block the activities of all sirtuins tested to date. Also, EX-527 and AGK2 are used, which specifically inhibit SIRT1 and SIRT2, respectively. All drug studies are optimized for both dose and time.

SIRT4 may repress fatty acid catabolism from both peroxisomes and mitochondria. If SIRT4 specifically affects mitochondrial fatty acid oxidation, identical rates of oxidation after treatment with etomoxir and mitochondrial inhibitors are observed. This result indicates that SIRT4 has the capacity to function in the regulation of beta oxidation by directly interacting with and repressing mitochondrial proteins involved in lipid uptake or catabolism.

### Example 11

### The Effect of SIRT4 on Mitochondrial Bioenergetics

Without being limited by theory, SIRT4 impacts electron transport and ATP production from amino acids and fatty acid catabolism. It has been shown that SIRT4 suppresses GDH enzymatic activity and regulates insulin secretion, a process highly dependent on mitochondrial function and ATP production. It is further demonstrated herein that SIRT4 regulates fatty acid oxidation. This aim proposes the next logical step: to perform a systematic analysis of how SIRT4 impacts mitochondrial bioenergetics in response to different nutrients. For these studies, we will use diverse approaches to analyze mitochondrial respiration, ATP production and ROS production. These experiments represent the first detailed and mechanistic study of SIRT4 function in mitochondrial bioenergetics.

We perform mitochondrial assays in primary MEFs (with varying levels of SIRT4) or primary hepatocytes from SIRT4 WT and SIRT4 KO liver, each of which is isolated using the methods described above. The effect of SIRT4 on mitochondrial respiration is examined using a Clarke-type oxygen electrode (Hansatech) these cells. The basal respiration from SIRT4 WT or KO MEFs or hepatocytes is analyzed by assaying for glucose, amino acids and/or fatty acids (palmitate or octanoate). Oligomycin is then added to inhibit coupled respiration, followed by the chemical uncoupler carbonyl cyanide P-(trifluoromethoxy) phenylhydrazone (FCCP) in order to determine the maximum possible rate of respiration that the mitochondria can support. Finally, KCN is added to inhibit mitochondrial respiration. Rotenone and antimycin A are used to inhibit complexes I and III, respectively. Rates are normalized to the protein content using the Bio-Rad Protein Assay kit.

To measure SIRT4 impact on the rate of mitochondrial respiration and the efficiency at which respiration is coupled to ATP production, the oxygen consumption of freshly isolated mitochondria is measured. Various substrates and inhibitors are used to differentiate the function of the five key complexes of the electron transport chain. Oxygen consumption is analyzed using a Clark oxygen electrode (Hansatech). Complex I respiration is measured using pyruvate, glutamate and/or malate as substrates is the presence or absence the specific inhibitor, rotenone. Complexes II+III use the substrate succinate; addition of antimycin inhibit complex III. Ascorbate is used as substrate for Complex IV and cyanide as the inhibitor. Palmitate, which requires fatty acid oxidation, is also used. Substrates are added to respiring mitochondria, with and without ADP, to measure respiration rates and determine the P/O ratio (molecules of ATP synthesized per 2e-transferred from substrates to ½ O₂). The P/O ratio, reflecting the degree of respiratory coupling, is calculated as the amount of ADP added, divided by the amount of oxygen used in the conversion of ADP to ATP. Function of complex V (H+-translocating ATP synthase) is determined by comparing respiration in the presence of ADP with or without an uncoupler (FCCP) present. These studies of the respiration of each component of the electron transport chain are sensitive measures of mitochondrial function that allow the identification of complexes affected by SIRT4.

The Seahorse XF24 Extracellular Flux Analyzer provides a complementary approach to analyzing mitochondrial function in living cells. The XF24 Analyzer simultaneously measures oxygen consumption rate (OCR) and extracellular acidification rate (ECAR) in a small number of intact cells. As most of the oxygen consumed by cells is used by mitochondria during electron transport, OCR is a good measure of mitochondrial respiration. Likewise, acidification of cell culture media is largely due to the production of lactic acid by glycolysis or pyruvate overload. Therefore, ECAR is a good measure of glycolysis or mitochondrial dysfunction. Lactic acid production also increases with mitochondrial dysfunction. SIRT4 WT or KO MEFs or hepatocytes (30,000 per well) are cultured in the wells of a specialty 24 well plate (embedded with oxygen and pH fluorescent biosensors, coupled to a fiber-optic waveguide and designed by Seahorse Bioscience). The day of the assay, cells are incubated in assay buffer (6 wells per genotype), containing non-buffered DMEM (supplied by Seahorse Bioscience). OCR and ECAR are recorded for basal rates, after the addition of the mitochondrial uncoupler 2,4-dinitrophenol (DNP, 100 mM), and after the addition of rotenone (or oligomycin) (1 mM). Studies are performed using palmitate, glucose or glutamine to drive respiration through pyruvate, fatty acid oxidation of amino acid metabolism. A significant advantage of the Seahorse XF24 Analyzer is its ability to measure oxygen consumption in 24 wells simultaneously without disturbing the normal environment of cultured cells; oxygen consumption is measured in intact cells attached to their normal culture vessel in a specialized culture plate. Because the cells remain viable one plate is analyzed, washed and then reanalyzed using a new set of substrates.

The effect of SIRT4 on mitochondrial ATP production is examined in SIRT4 WT or KO hepatocytes that have been incubated overnight in culture medium containing, e.g., 3 or 17 mM glucose, palmitate, or glutamine. ATP production is measured in living cells using a luciferase assay, which yields luminescence upon ATP hydrolysis (PerkinElmer). The samples are homogenized and centrifuged at 10,000 g for 15 min at 4° C. and the supernatant is collected for ATP analysis. The pellet is used for measurement of protein content. ATP measurements are performed in a luminometer (96-well plate reader to measure the reaction of ATP with luciferin at 562 nm. Standard ATP solution is used to construct a standard curve to calculate cellular ATP content. Standards and samples are analyzed in triplicate, and the results are expressed as nmol/mg protein. This experiment is also performed using inhibitors of mitochondrial respiration as a negative control.

The effects of SIRT4 on levels of key mitochondrial proteins such as cytochrome c, complex IV subunits I (encoded by mtDNA) and IV (encoded by nuclear DNA) determined with Western blots. The levels of other OXPHOS enzymes are measured depending on the results of the activity assays described herein. Mitochondrial lysates are prepared and analyzed by Western blot as described previously. Equal amounts of protein are loaded in each lane of an 8 or 16% Tris-glycine gel and separated by SDS-PAGE. Proteins are transferred to nitrocellulose membranes, incubated in blocking buffer, and treated with primary antibodies obtained from commercial sources or non-commercial antibodies available at the MAMMAG Center (UC-Irvine, in collaboration with Dr. Doug Wallace). Appropriate secondary antibodies are then applied, and protein bands are visualized using enhanced chemiluminescence reagent and Hyperfilm (GE Healthcare). Protein bands are identified based on predicted molecular weights and the position of positive control bands. Levels of mitochondrial porin also are measured on each blot to verify equal protein loading in every lane. UN-SCAN-IT software (Silk Scientific Inc., UT) is used for quantitative densitometric analysis of immunoreactive bands. The results from these mitochondrial studies reveal the effect of SIRT4 on mitochondrial respiration, lactic acid production as an indicator of glycolytic rate, ROS production, and the efficiency of ATP production. Because assays are performed using glucose, palmitate or glutamate as substrates, the data provide important mechanistic information about how ATP production from the metabolism of fats and amino acids is regulated by SIRT4. It is believe that loss of SIRT4 in the liver leads to increased oxidative respiration. Glutamine alteration of any of the measured mitochondrial functions in SIRT4 KO MEFs, indicates that GDH activity is involved; this is tested in cells deleted for SIRT4 and GDH using RNAi experiments, similar to studies performed in MIN6 cells. If data indicate that palmitate alters mitochondrial functions specifically in SIRT4 KO cells, PPAR-α activity is determined by treating cells with PPAR-α inhibitor MK886 or agonist WY14643. Interestingly, PPAR-a agonists mimic many effects of CR, one of which is to up-regulate liver β-oxidation and mitochondrial respiration. Data show SIRT4 interacts with ANT, which supplies ADP for ATP synthase. This represents a connection between mitochondrial respiration and fatty acid metabolism. Elevated amino acid and fatty acid catabolism lead to increased ketone body formation instead of or in addition to changes in mitochondrial bioenergetics. Ketones produced from fatty acids provide other tissues with energy during times of nutrient deprivation. Ketone production is measured from SIRT4 WT and KO primary hepatocytes using palmitate as the substrate.

### Example 12

### Identification of novel SIRT4 interacting proteins in hepatocytes

A SIRT4 complex is purified from a liver cell line, HepG2, to identify novel SIRT4 interacting proteins in hepatocytes that are directly involved in fatty acid metabolism and/or energy production.

HepG2 cell lines that maintain the stable expression of pCMV vector control, SIRT4-FLAG, or the H161Y SIRT4-FLAG variant are created. To generate stable lines, cells are transfected with control or SIRT4 plasmids, which contain neomycin resistance, and then selected using G418. Stable expression is verified by Western blotting using antibodies against the FLAG epitope.

To identify proteins that interact with SIRT4 in HepG2 cells, anti-FLAG immunoprecipitations are performed using SIRT4-FLAG or H161Y SIRT4-FLAG stable cells and using cells containing pCMV as a negative control. Briefly, cells are lysed in NP-40 buffer, and cleared lysates are incubated with resin conjugated to anti-M2 FLAG (Sigma). Then, resin is washed in NP-40 buffer and complexes will be eluted using FLAG peptide. All purification steps are performed in the cold room, in the presence of protease inhibitors, dithiothreitol (DTT), and phosphatase inhibitors. The elution is analyzed by SDS-PAGE, stained by Coomassie, followed by Mass spectrometry of bands unique to SIRT4 or H161Y SIRT4 (Taplin Mass Spectrometry Core Facility, Harvard Medical School). These experiments are generally repeated 3-5 times to determine the consistency of interaction. Interactions are verified by Western blotting elutions with antibodies.

Using the methods described above, a specific SIRT4-containing complex is purified. The active site variant, H161Y SIRT4, is used to stabilize interactions between SIRT4 and its substrates, compared with WT SIRT4, resulting in more bands in the H161Y SIRT4 elution. To reduce non-specific interactions complexes are immunoprecipitated from isolated mitochondria, instead of whole cell lysates. By starting with only 1000 mitochondrial proteins, the nonspecific binding of “sticky” cytosolic and nuclear proteins are eliminated. The washing step is optimized by increasing the salt concentration stepwise (from 150 mM to 300 mM) and adjusting the detergent for lysis. Also, tandem affinity purification using sequential immunoprecipitations of FLAG and HA epitopes are used.

To analyze SIRT4 interacting proteins, a SIRT1-7-FLAG IP is performed to test the sirtuin type specificity of these interacting protein. Particularly interesting are interactors that function directly in fatty acid metabolism and/or bioenergetics, because these interacting proteins provide insight for how SIRT4 regulates fatty acid oxidation. Once relevant interactions are verified by Western blotting, how their interactions with SIRT4 change with nutrient availability is examined. Finally, to test whether these interacting proteins are substrates of SIRT4, ADP-ribosylation assays using radioactive [³²P]-NAD, are performed as described herein.

### EQUIVALENTS

Those skilled in the art will recognize, or be able to ascertain using no more than routine experimentation, many equivalents of the specific embodiments of the invention described herein. Such equivalents are intended to be encompassed by the following claims.

1.-6. (canceled)

7. A method of treating or preventing a fatty acid oxidation disorder (FOD) in a mammalian subject, comprising administering to the subject an effective amount of an agent that reduces SIRT4 protein activity.

8. The method of claim 7, wherein the FOD is obesity, Medium Chain Acyl-CoA Dehydrogenase (MCAD) Deficiency, Short Chain Acyl-CoA Dehydrogenase (SCAD) Deficiency, long-chain Acyl-CoA dehydrogenase (LCAD) deficiency, Carnitine Palmityltransferase Translocase I & II Deficiency, Carnitine acylcarnitine translocase deficiency, Very Long Chain Acyl-CoA Dehydrogenase (VLCAD) Deficiency, Glutaricaciduria II, HMG Carnitine Transport Defect (Primary Carnitine Deficiency), Long Chain 3-Hydroxyacyl-CoA Dehydrogenase (LCHAD) Deficiency, Trifunctional Protein (TFP) Deficiency, 2,4 Dienoyl-CoA Reductase Deficiency, 3-Hydroxy Acyl CoA Dehydrogenase Deficiency (HADH), Electron Transfer Flavoprotein (ETF) Dehydrogenase Deficiency, steatosis or 3-Hydroxy-3 Methylglutaryl-CoA (HMG) Lyase Deficiency.

9. The method of claim 7, wherein the levels of SIRT4 are modulated in a hepatocyte.

10. (canceled)

11. The method of claim 7, wherein the agent comprises a nucleic acid that targets SIRT4 mRNA or an antibody that targets SIRT4 protein.

12. A method of evaluating the effect of a test compound on SIRT4, the method comprising: a) providing a reaction mixture comprising SIRT4 and a test compound; and b) evaluating a fatty acid oxidation activity of SIRT4.

13. The method of claim 12, wherein the test compound is a small molecule.

14. (canceled)

15. The method of claim 12, wherein the reaction mixture is provided in a eukaryotic cell.

16. The method of claim 15, wherein the cell is a hepatocyte.

17. The method of claim 12, wherein the reaction mixture is provided in a mammalian subject.

18.-19. (canceled)

20. The method of claim 7, wherein the agent increases an activity of a peroxisome proliferator-activated receptor-alpha (PPAR-a) in a mammalian cell.

21. A method of increasing a mammalian subject's energy consumption, comprising administering to the subject a SIRT4 inhibitor.

22. The method of claim 21, wherein the subject is overweight.

23. The method of claim 21, wherein the SIRT4 inhibitor is provided in an effective dose such that fat storage in a tissue of the subject is reduced.

24. The method of claim 21, comprising administering the SIRT4 inhibitor to a liver tissue, a brown adipose tissue or a skeletal muscle tissue.

25. The method of claim 21, wherein the subject is suffering from or at risk of developing a mitochondrial-related disease.

26. The method of claim 25, wherein the mitochondrial-related disease is selected from the group consisting of aging, MELAS syndrome, muscular dystrophy, diabetes, Leber's hereditary optic neuropathy, Leigh syndrome, NARP syndrome, and Myoneurogenic gastrointestinal encephalopathy.

27.-30. (canceled)

31. A composition comprising a SIRT4 inhibitor and a peroxisome proliferator-activated receptor-alpha agonist.

32.-37. (canceled)

38. The method of claim 12, wherein the reaction mixture is cell-free reaction mixture.


SEQUENCE LISTING (2 sequences)

SEQ ID NO: 1 - protein, 314 aa, Homo sapiens
MKMSFALTFRSAKGRWIANPSQPCSKASIGLFVPASPPLDPEKVKELQRFITLSKRLLVMTGAGISTESGIPDYRSEKVGLYARTDRRPIQHGDFVRSAPIRQRYWARNFVGWPQFSSHQPNPAHWALSTWEKLGKLYWLVTQNVDALHTKAGSRRLTELHGCMDRVLCLDCGEQTPRGVLQERFQVLNPTWSAEAHGLAPDGDVFLSEEQVRSFQVPTCVQCGGHLKPDVVFFGDTVNPDKVDFVHKRVKEADSLLVVGSSLQVYSGYRFILTAWEKKLPIAILNIGPTRSDDLACLKLNSRCGELLPLIDPC

SEQ ID NO: 2 - protein, 333 aa, Mus musculus
MSGLTFRPTKGRWITHLSRPRSCGPSGLFVPPSPPLDPEKIKELQRFISLSKKLLVMTGAGISTESGIPDYRSEKVGLYARTDRRPIQHIDFVRSAPVRQRYWARNFVGWPQFSSHQPNPAHWALSNWERLGKLHWLVTQNVDALHSKAGSQRLTELHGCMHRVLCLNCGEQTARRVLQERFQALNPSWSAEAQGVAPDGDVFLTEEQVRSFQVPCCDRCGGPLKPDVVFFGDTVNPDKVDFVHRRVKEADSLLVVGSSLQVYSGYRFILTAREQKLPIAILNIGPTRSDDLACLKLDSRCGELLPLIDPRRQHSDVQRLEMNFPLSSAAQDP